SEQUENCE LISTING (4 sequences)

SEQ ID NO: 1 - DNA, 24 bp, Artificial sequence
  Primer IL-2F
ctaggccacagaattgaaagatct

SEQ ID NO: 2 - DNA, 25 bp, Artificial sequence
  Primer IL-2R
gtaggtggaaattctagcatcatcc

SEQ ID NO: 3 - DNA, 28 bp, Artificial sequence
  Primer HD82Q-F
gtggataccccctcccccagcctagacc

SEQ ID NO: 4 - DNA, 29 bp, Artificial sequence
  Primer HD591-5′R
gaactttcagctaccaagaaagaccgtgt

(19) United States
(12) Patent Application Publication
(10) Pub. No.: US 2007/0155771 A1
(43) Pub. Date: Jul. 5, 2007

(54) Methods and means for treating protein conformational disorders

(76) Inventors: David Rubinsztein, Cambridge (GB); Brinda Ravikumar, Cambridge (GB); Julie Webb, Cambridge (GB)

Correspondence Address: NIXON & VANDERHYE, PC, 901 NORTH GLEBE ROAD, 11TH FLOOR, ARLINGTON, VA, 22203, US

(21) Appl. No.: 10/553,262

(22) PCT Filed: Feb. 24, 2004
(86) PCT No.: PCT/GB04/00690

§ 371 Date: Dec. 20, 2005

Related U.S. Application Data
(60) Provisional application No. 60/462,269, filed on Apr. 11, 2003.

(51) Int. Cl. A61K 31/4745

(52) U.S. Cl. 514/291

(57) ABSTRACT

This invention relates to the recognition that autophagy plays a key role in the clearance of the intracellular protein aggregates which characterise Protein Conformational Disorders, such as Huntington's disease and Parkinson's disease. Methods and uses of autophagy inducing agents, such as rapamycin macrolides, in the treatment of Protein Conformational Disorders, are described herein.

This invention relates to compositions and methods for the treatment of a range of conditions that are characterised by the formation of intracellular protein aggregates (known as Protein Conformational Disorders), including Huntington's disease.

Proteinopathies or Protein Conformational Disorders (PCDs) are associated with particular proteins or sets of proteins that misfold and aggregate in specific tissues (Paulson, H. L. (1999) Am. J. Hum. Genet. 64 339-345). Some PCDs are caused by codon reiteration mutations, where protein misfolding is mediated by the abnormal expansion of a tract of repeated amino acids. These include polyglutamine (polyQ) expansion diseases, exemplified by Huntington's disease (HD). HD is characterised by expansions of a polyQ stretch in exon 1 of the Huntington gene to more than 37 glutamines, and a short N-terminal fragment encoding the polyglutamine stretch is sufficient to cause aggregates in mice (Schilling, G. et al. (1999) Hum. Mol. Genet. 8 397-407) and in cell models (Wyttenbach, A. et al (2000) Proc. Natl. Acad. Sci. USA 97 2898-2903). It is commonly believed that the mutant protein acquires its toxicity and its propensity to aggregate after cleavage, forming a short (so far, incompletely-defined) N-terminal fragment containing the polyglutamine stretch (Martindale, D. et al., (1998) Nat. Genet., 18, 150-154). Recently, polyalanine (polyA) expansion mutations in the polyadenine-binding protein 2 gene have been shown to cause OPMD, which is associated with aggregates in muscle cell nuclei (Brais, B et al. (1998). Nat. Genet. 18, 164-167). This disease has been modelled in cell culture systems where aggregate formation is associated with cell death (Fan, X et al (2001) Hum. Mol. Genet. 10, 2341-2351) PolyA expansions of 19. or more repeats tagged with enhanced green fluorescent protein are sufficient to cause intracytoplasmic aggregate formation and cell death in cultured cells (Rankin, J. et al (2000) Biochem. J., 348, 15-19). Many of the codon reiteration diseases are dominantly inherited and genetic and transgenic studies suggest that they are generally due to gain-of-function mutations (for instance in polyQ diseases) (Narain, Y. et al (1999). J. Med. Genet., 36, 739-746).

Parkinsons disease (PD) is caused by the degeneration of dopaminergic neurons in the substantia nigra. The pathogenic hallmark of PD is the accumulation and aggregation of α-synuclein in susceptible neurons. The cytoplasmic aggregates/inclusions characteristic of PD are called Lewy bodies and their major constituent is α-synuclein (Kahle, P. J. et al (2002) J. Neurochem. 82, 449457). Lewy pathology is also found in dementia with Lewy Bodies (LB), the LB variant of Alzheimer's disease, in neurodegeneration with brain iron accumulation type I and in glial cytoplasmic inclusions of multiple system atrophy. These diseases are collectively known as α-Synucleinopathies (Spillantini, M. G. et al (1997) Nature 388, 839840, Mezey, E et al. (1998) Mol. Psychiatry 3, 493499).

In many cases, disease severity correlates with the expression levels of the mutant protein. Factors regulating clearance of these aggregate-prone proteins and of the aggregates themselves may therefore play an important role in the disease. Aggregate-prone proteins are generally believed to be cleared by the ubiquitin-proteasome pathway, where malfolded proteins are tagged with ubiquitin, which serves to route them to the proteasome for degradation. The role of this pathway in polyQ diseases is supported by the observations that these proteins are ubiquitinated and that the aggregates are associated with proteasomal subunits (Wyttenbach et al (2000) supra; Jana, N. R. et al (2001) Hum. Mol. Genet. 10, 1049-59; Suhr S. T. et al (2001) J. Cell Biol. 153, 283-294). Furthermore, inhibition of the proteasome with lactacystin, or its more potent derivative beta-lactone, resulted in increased inclusion formation (Wyttenbach et al (2000) supra; Waelter, S. et al (2001) Mol. Biol. Cell, 12, 1393-1407; Bence, N. F. et al (2001) Science, 292, 1552-1555.).

The lysosomes, which are often considered as non-specific systems for protein degradation, have recently been shown to be able to selectively receive and degrade certain-intracellular proteins. The process of bulk degradation of cytoplasmic proteins or organelles in the lytic compartment is termed autophagy. It involves the formation of double membrane structures called autophagosomes or autophagic vacuoles, which fuse with the primary lysosomes where their contents are degraded and then either disposed off or recycled back to the cell (Klionsky, D. J. and Oshumi, Y. (1999) Annu. Rev. Cell Dev. Biol. 15, 1-32).

The present inventors have recognised that the autophagy-lysosome pathway is a major route for the degradation of aggregate-prone proteins and aggregates. Inhibition of autophagy increased the levels and rate of aggregate formation, while increased aggregate clearance was associated with the stimulation of autophagy by rapamycin. This finding has significant application in the treatment of Protein Conformational Disorders, in which the formation of aggregates is closely associated with toxicity.

Various aspects of the invention relate to methods and means for the treatment of protein conformational disorders, including codon reiteration mutation disorders and α-synucleinopathies, by stimulation of autophagic activity.

An aspect of the invention provides a method of treating a protein conformational disorder in an individual comprising: stimulating autophagy activity in the cells of the individual.

Protein conformational disorders are characterised by the intracellular accumulation of protein aggregates. Aggregates may accumulate, for example, in the cytoplasm of a cell. Commonly, aggregates may form in neuronal cells, such as brain cells, for example in disorders such as Huntington's disease and Parkinson's disease.

Protein conformational disorders which may be treated in accordance with the invention include codon reiteration mutation disorders, in particular polyQ expansion disorders such as Huntington's disease, spinocerebellar ataxias types 1, 2, 3, 6, 7, and 17, Kennedy's disease and dentatorubral-pallidoluysian atrophy. These disorders are characterised by the aggregation of mutant proteins which contain an expanded tract of repeated glutamine residues. For example, HD is characterised by an expanded polyQ stretch in exon 1 of the Huntington gene.

Protein conformational disorders which may be treated in accordance with the invention also include polyA expansion disorders. These disorders are characterised by the aggregation of mutant proteins which contain an expanded tract of repeated alanine residues. For example, oculapharyngeal muscular dystrophy (OPMD) is characterised by a polyadenine (polyA) expansion mutation in the polyadenine binding protein 2 gene.

Other protein conformational disorders which may be treated in accordance with the invention may include α-synucleiopathies such as Parkinson's disease, LB variant Alheimer's disease and LB dementia. These are disorders characterised by the accumulation of cytoplasmic aggregates called Lewy bodies, which comprise α-synuclein.

Protein conformational disorders that may be treated in accordance with the invention also include prion disorders such as CJD.

The enhancement of the autophagic/lysosomal pathway is shown herein to mediate the clearance of protein aggregates, in particular cytoplasmic protein aggregates, which are associated with toxicity in protein conformational disorders.

Various methods of stimulating the activity of the autophagic/lysosmal pathway are possible. For example, an individual may be subjected to a leucine-free dietary regime or an agent or composition which stimulates autophagy may be administered.

In preferred embodiments, activity may be stimulated or enhanced in the cells of the individual by administering an autophagy-inducing agent to said individual.

An autophagy-inducing agent may be any compound or molecule which stimulates or induces the activity of the autophagic/lysosomal pathway within cells. Particularly suitable autophagy inducing agents include rapamycin macrolides such as rapamycin and its numerous analogues and derivatives.

Rapamycin and its derivatives and analogues are lactam macrolides. A macrolide is a macrocyclic lactone, for example a compound having a 12-membered or larger lactone ring. Lactam macrolides are macrocyclic compounds which have a lactam (amide) bond in the macrocycle in addition to a lactone (ester) bond.

Rapamycin is produced by Streptomyces hygroscopicus, and has the structure shown below.

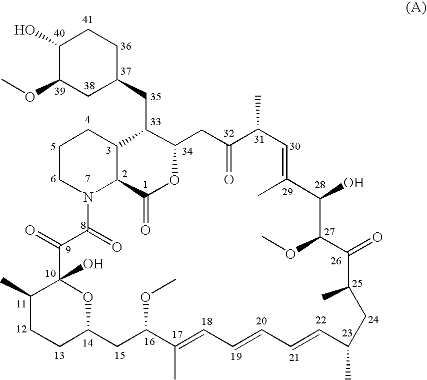

See, e.g., McAlpine J. B. et al. J. Antibiotics (1991) 44: 688; Schreiber, S. L. et al. J. Am. Chem. Soc. (1991) 113:7433; U.S. Pat. No. 3,929,992.

One group of rapamycin analogues are 40-0-substituted derivatives of rapamycin having the structure set out below; 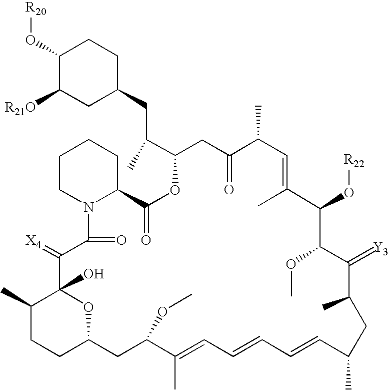
wherein; X₄ is (H, H) or O; Y₃ is (H, OH) or O; R₂₀ and R₂₁ are independently selected from H, alkyl, arylalkyl, hydroxyalkyl, dihydroxyalkyl, hydroxyalkoxycarbonylalkyl, hydroxyalkylaryalkyl, dihydroxyalkylarylalkyl, acyloxyalkyl, aminoalkyl, alkylaminoalkyl, alkoxycarbonylaminoalkyl, acylaminoalkyl, arylsulfonamidoalkyl, allyl, dihydroxyalkylallyl, dioxolanylallyl, dialkyl-dioxolanylalkyl, di(alkoxycarbonyl)-triazolyl-alkyl and hydroxyalkoxy-alkyl;
wherein “alk-” or “alkyl” refers to C₁₋₆ alkyl, branched or linear, preferably C₁₋₃ alkyl,; “aryl” is phenyl or tolyl; and acyl is a radical derived from a carboxylic acid; and;
R₂₂ is methyl or R₂₂ and R₂₀ together form C₂₋₆ alkyl; provided that R₂₀ and R₂₁ are not both H; and hydroxyalkoxyalkyl is other than hydroxyalkoxymethyl.

Suitable rapamycin analogues are disclosed in WO 94/09010 and WO 96/41807.

Particularly suitable rapamycin analogues include 40-O-(2-hydroxy)ethyl-rapamycin, 32-deoxo-rapamycin, 16-O-pent-2-ynyl-32-deoxo-rapamycin, 16-O-pent-2-ynyl-32-deoxo-40-O-(2-hydroxyethyl)-rapamycin, 16-O-pent-2-ynyl-32-(S)-dihydro-rapamycin and 16-O-pent-2-ynyl-32-(S)-dihydro-40-O-(2-hydroxyethyl)-rapamycin.

Other rapamycin analogues include hydroxyesters of rapamycin, such as 3-hydroxy-2-(hydroxymethyl)-2-methylpropionic acid (CCI-779). The preparation and use of hydroxyesters of rapamycin, including CCI-779, are disclosed in U.S. Pat. No. 5,362,718 and 6,277,983

Other rapamycin analogues include carboxylic acid esters as set out in WO 92/05179, amide esters as set out in U.S. Pat. No. 5,118,677, carbamates as set out in U.S. Pat. No. 5,118,678, fluorinated esters as set out in U.S. Pat. No. 5,100,883, acetals as set out in U.S. Pat. No. 5,151,413, silyl ethers as set out in U.S. Pat. No. 5,120,842 and arylsulfonates and sulfamates as set out in U.S. Pat. No. 5,177,203.

Other rapamycin analogues which may be used in accordance with the invention may have the methoxy group at the position 16 replaced with alkynyloxy as set out in WO 95/16691. Rapamycin analogues are also disclosed in WO 93/11130, WO 94/02136, WO 94/02385 and WO 95/14023.

Rapamycin and its analogues are known to possess immunsuppressive activity and are in clinical use for the treatment of transplant rejection (Chan, L. et al (2001) Am. J. Kidney Dis., 38, S2-S9).

Another aspect of the invention provides the use of an autophagy inducing agent in the manufacture of a medicament for use in the treatment of a protein conformational disorder.

Protein conformational disorders and autophagy inducing agents are described in detail above.

Another aspect of the invention provides a pharmaceutical composition for use in the treatment of a protein conformational disorder comprising a rapamycin macrolide and a pharmaceutically acceptable excipient, vehicle or carrier.

A pharmaceutically acceptable excipient, vehicle or carrier, should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Examples of techniques and protocols mentioned above can be found in Remington's Pharmaceutical Sciences, 16th edition, Osol, A. (ed), 1980.

Formulations and administration regimes which are suitable for use with rapamycin macrolides such as rapamycin are well known in the art.

Administration is preferably in a “therapeutically effective amount”, this being sufficient to show benefit to the individual. The actual amount administered, and rate and time-course of administration, will depend on the nature and severity of what is being treated. Prescription of treatment, e.g. decisions on dosage etc, is within the responsibility of medical practitioners.

A composition may be administered alone or in combination with other treatments, either simultaneously or sequentially dependent upon the condition to be treated

Other aspects of the invention provide screening methods for agents useful in treating protein conformational disorders.

A method of identifying an agent useful in the treatment of a protein conformational disorder may comprise;
contacting a mammalian cell with a test compound; and,
determining the autophagy activity of said cell, an increase in autophagy activity in the presence of said compound being indicative that the compound is a candidate agent for use in the treatment of a protein conformational disorder.

A method may include determining the ability of said compound to increase the clearance of cytoplasmic protein aggregates. This may be determined for example, in the presence of proteosome inhibitors such as epoximicin.

Any suitable mammalian cell may be used, for example Chinese hamster ovary cells, baby hamster kidney cells, COS cells, PC12 and many others.

In some embodiments, a cell for use in the present methods may comprise a heterologous nucleic acid encoding an aggregation-prone polypeptide, for example α-synuclein, huntingtin, or polyadenine-binding protein 2. An aggregation-prone polypeptide may comprise an aggregation-inducing mutation, for example a codon iteration mutation such as a polyQ or a polyA insertion, or may have the non-mutant, wild-type sequence. Clearance of the encoded aggregation-prone polypeptide, either in an aggregated or a soluble monomeric form, may be determined.

In some embodiments, expression of the heterologous nucleic acid may be reversible i.e. expression may be induced and repressed as required, for example by adding or removing an inducer compound. A method of the invention may comprise inducing and repressing the expression of said nucleic acid prior to contacting the mammalian cell with the test compound. Many examples of inducible and/or reversible expression systems and constructs are known in the art, including, for example the Tet-on™ expression (Clontech), in particular in combination with the pTet-tTs™ vector (Clontech). Autophagy activity may be determined by any convenient method, for example monodansylcadaverine (MDC) staining (Ravikumar et al Hu. Mol. Gen. (2003) 12 9 1-10), LC3 processing (Y. Kabeya et al EMBO J. 19 5720-5728), or electron microscopy visulating autophagosome numbers.

Compounds which increase or induce autophagy may include compounds which inhibit the activity of mammalian TOR (Target of Rapamycin: Schmelzle, T. & Hall, M. N. Cell 103, 253-262 (2000)). In some embodiments, a method may comprise contacting the test compound with an mTOR-polypeptide and determining the inhibition of mTOR activity by the test compound.

Methods of determining mTOR activity are well known in the art and exemplified herein.

Test compounds may be natural or synthetic chemical compounds used in drug screening programmes. Extracts of plants which contain several characterised or uncharacterised components may also be used.

Combinatorial library technology (Schultz, J S (1996) Biotechnol. Prog. 12: 729-743) provides an efficient way of testing a potentially vast number of different substances for ability to modulate activity of a polypeptide. Suitable test compounds may be based on rapamycin i.e. rapamycin derivatives or analogues.

The amount of test substance or compound which may be added to an assay will normally be determined by trial and error depending upon the type of compound used. Typically, from about 0.1 to 100 μM concentrations of putative inhibitor compound may be used, for example from 1 to 10 μM. The test substance or compound is desirably membrane permeable in order to access the intracellular components of the autophagy/lysosome pathway.

Following identification of a compound as described above, a method may further comprise modifying the compound to optimise the pharmaceutical properties thereof.

Such a method may comprise determining the autophagy inducing activity of the modified compound.

For example, a-method of producing an agent for the treatment of a protein conformational disorder may comprise;
modifying a rapamycin macrolide, such as rapamycin, to produce a derivative; and,
determining the autophagy inducing activity of said derivative.

A method may comprise determining the mTOR inhibiting activity of said derivative.

The modification of a ‘lead’ compound identified as biologically active is a known approach to the development of pharmaceuticals. Modification of a known active compound (such as rapamycin) may be used to avoid randomly screening large number of molecules for a target property.

Modification of a ‘lead’ compound to optimise its pharmaceutical properties commonly comprises several steps. Firstly, the particular parts of the compound that are critical and/or important in determining the target property are determined. These parts or residues constituting the active region of the compound are known as its “pharmacophore”.

Once the pharmacophore has been found; its structure is modelled to according its physical properties, e.g. stereochemistry, bonding, size and/or charge., using data from a range of sources, e.g. spectroscopic techniques, X-ray diffraction data and NMR.

Computational analysis, similarity mapping (which models the charge and/or volume of a pharmacophore, rather than the bonding between atoms) and other techniques can be used in this modelling process.

A template molecule is then selected onto which chemical groups which mimic the pharmacophore can be grafted. The template molecule and the chemical groups grafted on to it can conveniently be selected so that the modified compound is easy to synthesise, is likely to be pharmacologically acceptable, and does not degrade in vivo, while retaining the autophagy inducing activity of the lead compound. The modified compounds found by this approach can then be screened to see whether they have the target property, or to what extent they exhibit it. Modified compounds include mimetics of the lead compound.

Further optimisation or modification can then be carried out to arrive at one or more final compounds for in vivo or clinical testing.

The test compound may be manufactured and/or used in preparation, i.e. manufacture or formulation, of a composition such as a medicament, pharmaceutical composition or drug. These may be administered to individuals, e.g. for any of the purposes discussed elsewhere herein.

A method of the invention may comprise formulating said test compound in a pharmaceutical composition with a pharmaceutically acceptable excipient, vehicle or carrier as discussed further below.

Another aspect of the present invention provides a method of producing a pharmaceutical composition for use in the treatment of a protein conformational disorder comprising;
i) identifying a compound as an agent which increases autophagy activity in a cell using a method described herein; and,
ii) admixing the compound identified thereby with a pharmaceutically acceptable carrier.

The formulation of compositions with pharmaceutically acceptable carriers is described further below.

Another aspect of the invention provides a method for preparing a pharmaceutical composition, for example, for the treatment of a protein conformational disorder as described herein, comprising;
i) identifying a compound which increases autophagy activity in a cell,
ii) synthesising the identified compound, and;
iii) incorporating the compound into a pharmaceutical composition.

The identified compound may be synthesised using conventional chemical synthesis methodologies. Methods for the development and optimisation of synthetic routes are well known to persons skilled in this field.

The compound may be modified and/or optimised as described above.

Incorporating the compound into a pharmaceutical composition may include admixing the synthesised compound with a pharmaceutically acceptable carrier or excipient.

Administration of an autophagy inducing compound, for example a rapamycin macrolide as described herein, is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

A composition may be administered alone or in combination with other treatments, either simultaneously or sequentially dependent upon the condition to be treated.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, or Lactated Ringer's Injection. Preservatives, stabilisers, buffers, antioxidants and/or other additives may be included, as required.

Various further aspects and embodiments of the present invention will be apparent to those skilled in the art in view of the present disclosure. All documents mentioned in this specification are incorporated herein by reference in their entirety.

Certain aspects and embodiments of the invention will now be illustrated by way of example and with reference to the figures described below.

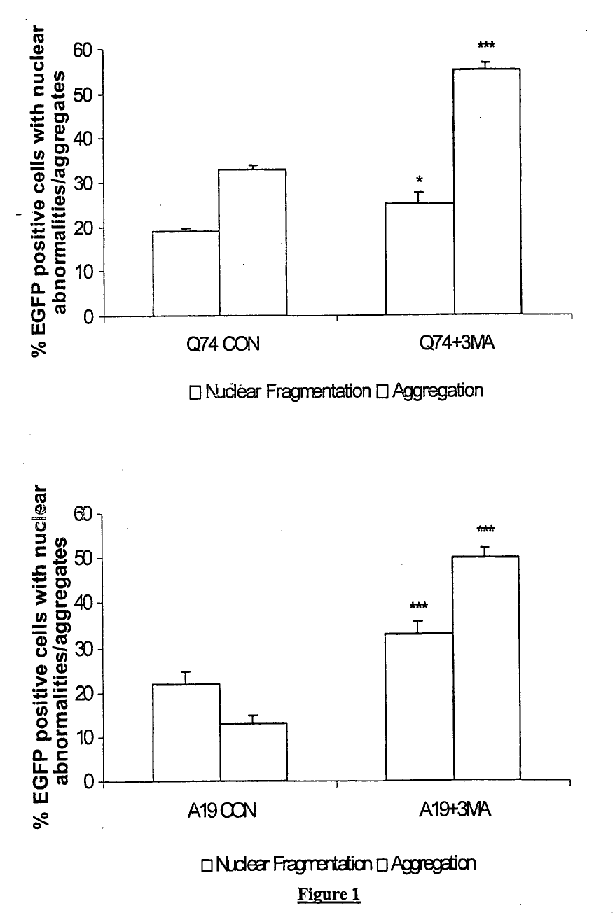

FIG. 1 shows the increases in aggregation and cell death caused by 3-methyl adenine (3-MA) in transient transfections COS-7 cells expressing an exon 1 fragment of the HD gene with 74 glutamines (Q74) or enhanced green fluorescent protein (EGFP) fused to 19 alanines (A19). The percentages of Q74- or A19-transfected COS-7 cells with aggregates and abnormal nuclei are shown 48 hours after transfection with Q74 (top) or A19 (bottom) without (−) or with (+) treatment with 10 mM 3-MA for 15 hours prior to fixing.

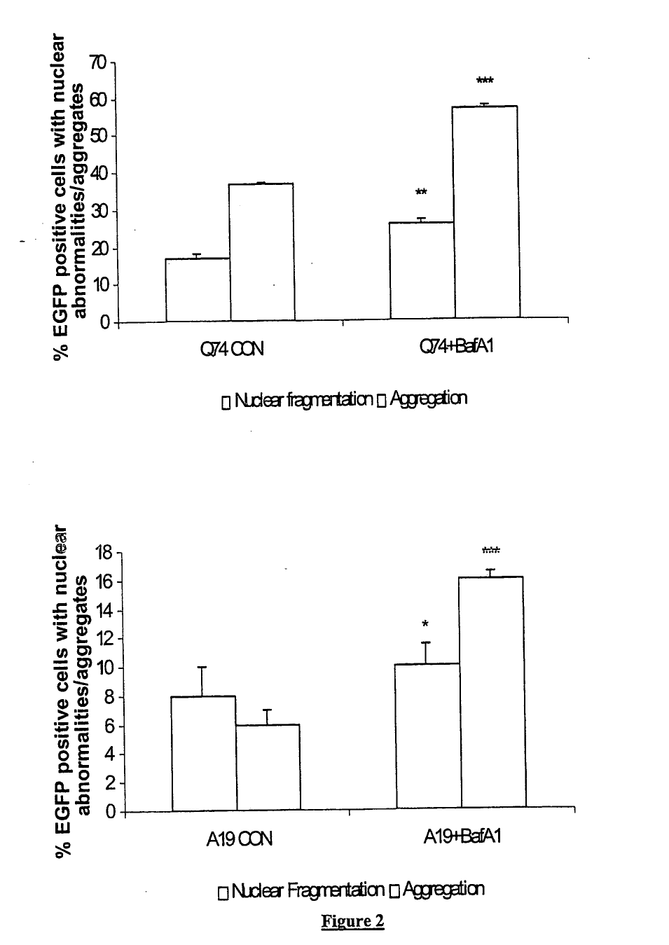

FIG. 2 shows that Bafilomycin A1 (BafA1) increases aggregation and cell death in COS-7 cells expressing Q74 or A19. The percentages of EGFP-positive cells with Q74 or A19 aggregates and abnormal nuclear morphology are shown after 48 hours of transfection with (+) or without (−) 200 nM BafA1. BafA1 was added 15 h before fixing cells. CON=control, BafA1=Bafilomycin A1.

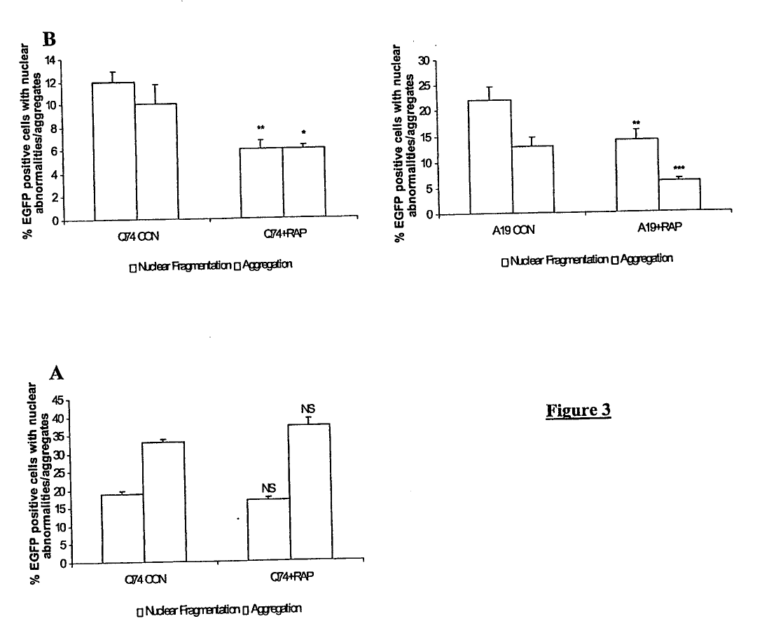

FIG. 3 shows that Rapamycin reduces aggregation and cell death in COS-7 cells expressing Q74 or A19. FIG. 3A shows the percentages of Q74 or A19 transfected COS-7 cells after 48 hours with aggregates and abnormal nuclear morphology. CON=control; RAP=rapamycin (at 0.2 μg/ml final concentration added 15 hours prior to fixing). FIG. 3B shows the percentages of EGFP-positive COS-7 cells with Q74 aggregates and cell death after 24 hours of transfection. Here rapamycin was added as in A.

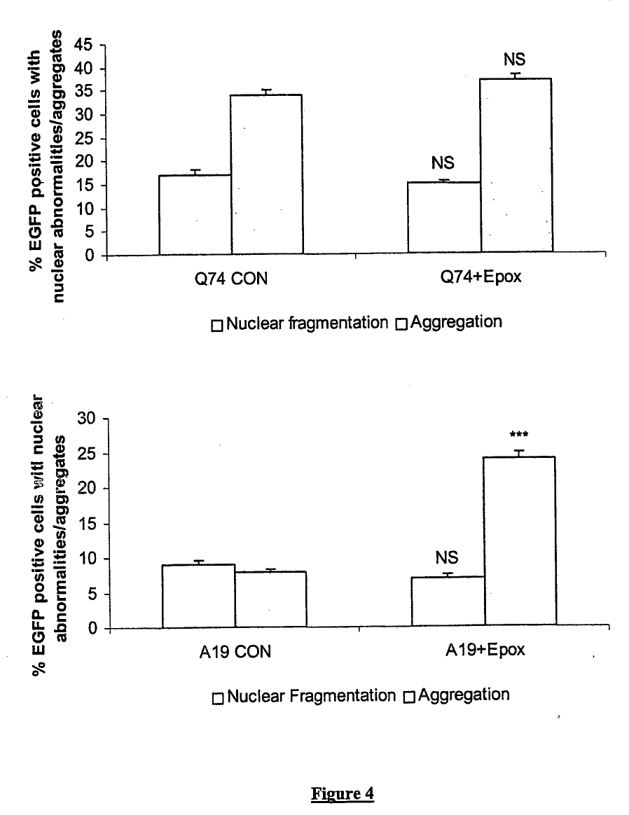

FIG. 4 shows that the effect of epoxomicin on COS-7 cells transfected with Q74 or A19. The percent of Q74- or A19-transfected cells with aggregates and nuclear abnormalities is shown with and without 10 μM epoxomicin added 15 hours before fixing the cells. CON=control, Epox=epoxomicin.

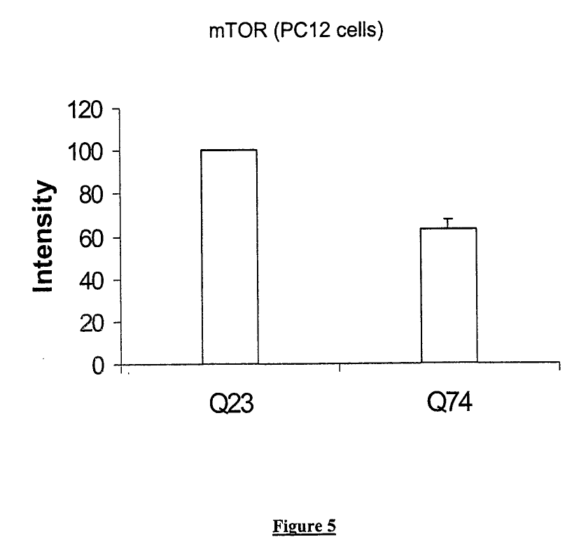

FIG. 5 shows a quantitation of western blots using lysates from Q23- or Q74-expressing stable inducible PC12 cells which show s reduced level of soluble mTOR (as a function of tubulin) in Q74 as compared to Q23 cells.

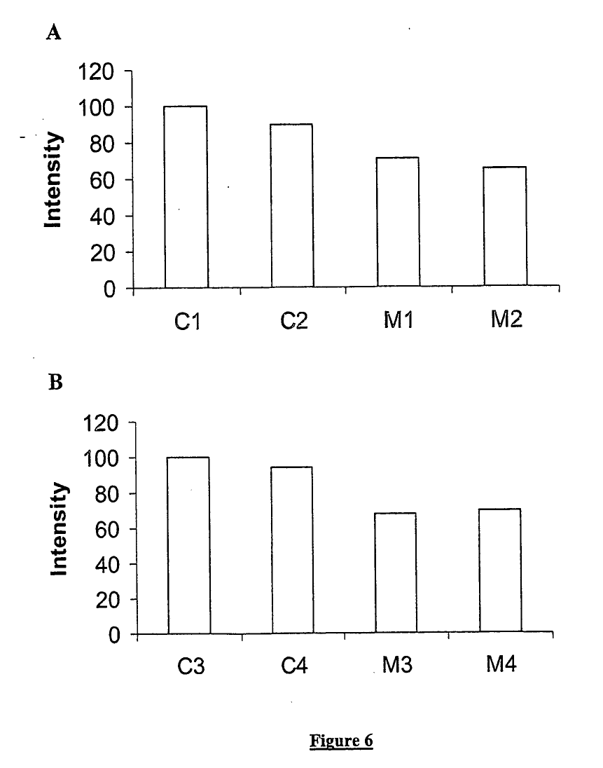

FIG. 6A shows a quantitation of western blot data showing that reduced levels of soluble mTOR can be seen in brain lysates from HD transgenic (N-terminal 1-171 of huntingtin with 82 glutamine repeats) mice (28 weeks-M1, M2) when compared to wild-type mice (28 weeks-C1, C2).

FIG. 6B shows a quantitation of western blot data showing that reduced levels of soluble mTOR can be seen in brain lysates from HD transgenic (N-terminal 1-171 of huntingtin with 82 glutamine repeats) mice (24 weeks-M3, M4) when compared to wild-type mice (24 weeks-C3, C4).

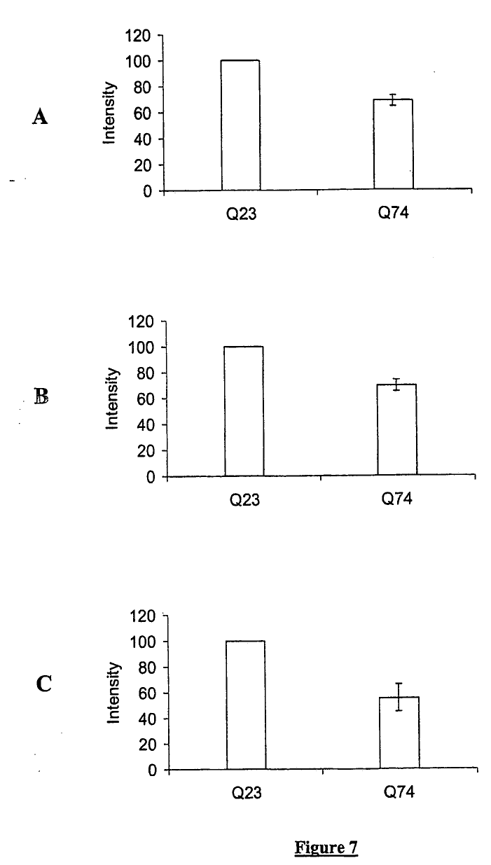

FIG. 7A shows a quantitation of a western blot analysis using cell lysates from wild-type (Q23)- or mutant (Q74)-expressing stable inducible PC12 cells. Intensity of phospho-S6K1/total-S6K1 in Q74 as a percentage of the same parameter in Q23 is shown. Reduced levels of phospho-S6K1 are shown as a function of total S6K1 in Q74 compared to Q23 after 48 h of induction of the transgene.

FIG. 7B shows a quantitation of a western blot analysis using cell lysates from wild-type (Q23)- or mutant (Q74)-expressing stable inducible PC12 cells. Intensity of phospho-4E-BP1/total-4E-BP1 in Q74 as a percentage of the same parameter in Q23 is shown below the gel. Reduced levels of phospho-4E-BP1 are shown as a function of total-4E-BP1 in Q74 compared to Q23 after 48 h of induction of the transgene.

FIG. 7C shows a guantitation of a western blot analysis of the phosphorylation of transiently transfected FLAG-tagged 4E-BP1. Intensity of phospho-4E-BP1/total-4E-BP1 in Q74. as a percentage of the same parameter in Q23 is shown. Reduced levels of phosphorylated 4E-BP1 in Q74 compared to Q23 are shown.

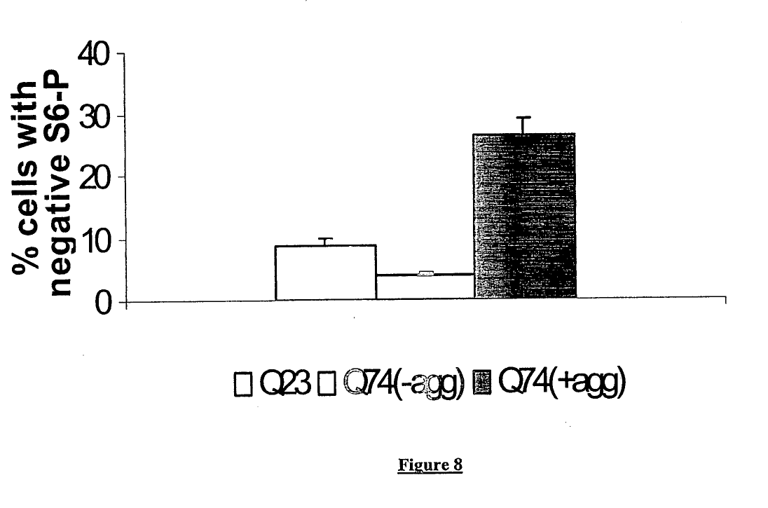

FIG. 8 shows an analysis of COS-7 cells transiently transfected with Q23 or Q74 and labelled for S6 phosphorylated at Ser 235 and 236. Wild-type (Q23, Q25) and mutant cells (Q74, Q103) with (+agg) and without (agg) aggregates were scored for negative phospho-S6 staining, showing that a significant proportion of mutant cells with aggregates stained negative for phospho-S6.

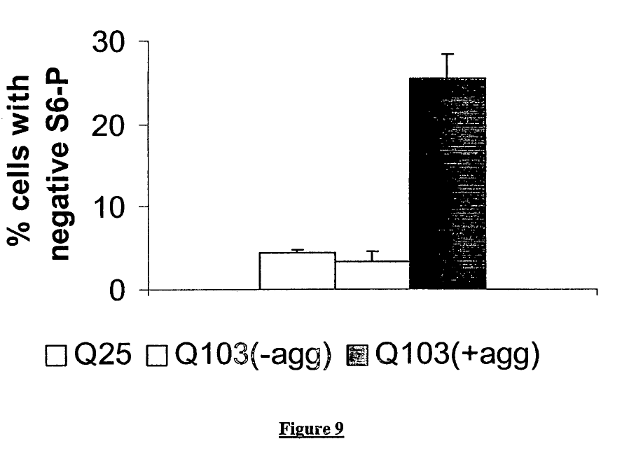

FIG. 9 shows an analysis of COS-7 cells transiently transfected with huntingtin exon-1 constructs with 25 (Q25) or 103(Q103) glutamine repeats and labelled for S6 phosphorylated at Ser 235 and 236. Wild-type (Q23, Q25) and mutant cells (Q74, Q103) with (+agg) and without (agg) aggregates were scored for negative phospho-S6 staining, showing that a significant proportion of mutant cells with aggregates stained negative for phospho-S6.

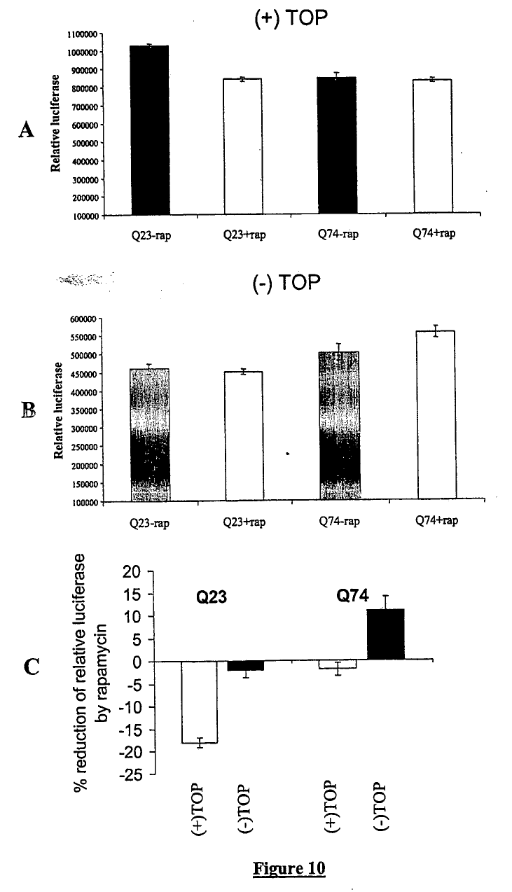

FIG. 10 shows an analysis of PC12 cells stably expressing Q23 or Q74 were transfected with luciferase construct having a 5′-untranslated region from eEF2 gene containing a terminal oligopyrimidine tract (TOP-luciferase) (FIG. 10A) or without TOP sequence (Control-luciferase) (FIG. 10B) along with β-galactosidase to control transfection efficiency. Data are shown for one representative experiment in sextuplicate. The experiment was repeated three times and showed similar trend. Raw data is shown in FIGS. 10A and 10B the percent reduction of relative luciferase by rapamycin is shown in FIG. 10C.

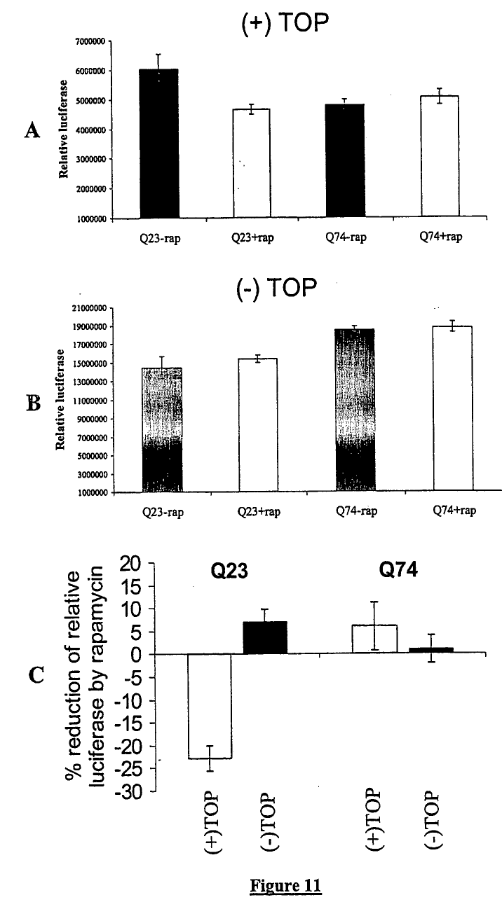

FIG. 11 shows an analysis of COS-7 cells co-transfected with Q23 or Q74 and a different non-typical TOP-luciferase construct having a 5′-untranslated region from eEF2 gene containing a terminal oligopyrimidine tract (TOP-luciferase) (FIG. 11A) or without TOP sequence (Control-luciferase) (FIG. 11B) along with β-galactosidase to control transfection efficiency. Data are shown for one representative experiment in sextuplicate. The experiment was repeated three times and showed similar trend. Raw data is shown in FIGS. 11A and 11B the percent reduction of relative luciferase by rapamycin is shown in FIG. 11C.

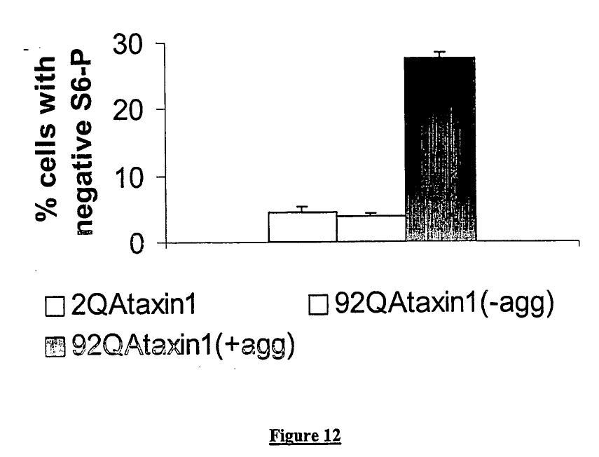

FIG. 12 shows a quantification of COS-7 cells transfected with wild-type ataxin 1 containing 2 glutamine repeats (2Q Ataxin) without aggregates and 92 glutamine repeats with and without aggregates for phospho-S6 staining, showing that 28% (+/−4.7) of cells with aggregates showed negative phospho-S6 staining. Data were analysed with ANOVA.

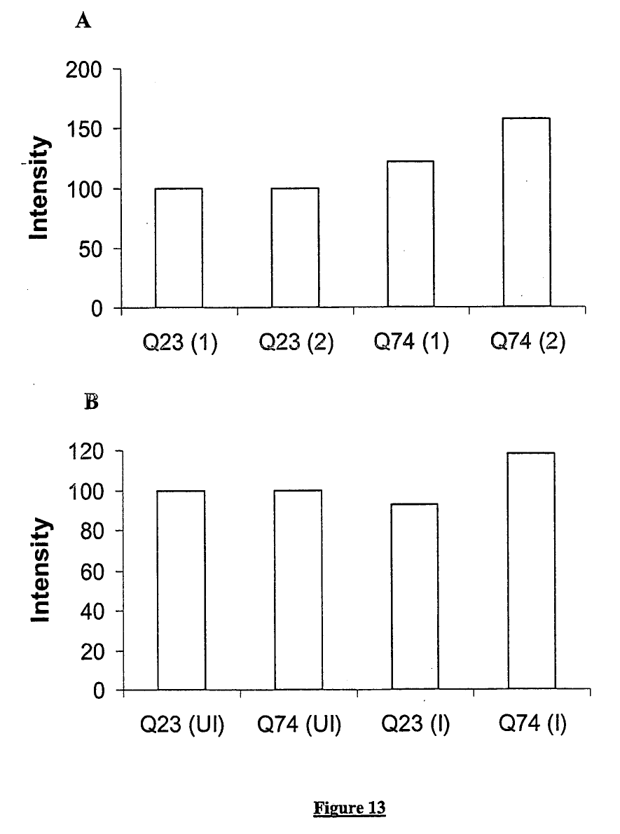

FIG. 13 shows an analysis of LC3-II levels in mutant huntingtin lines. LC3-II levels, which closely correlate with autophagic activity, increased in our mutant lines (Q74) compared to wild-type (Q23) as a function of actin (FIG. 13A). LC3-II levels in the mutant line (Q74) increased upon induction of the transgene (I), compared to uninduced (UI) wild-type (Q23) or mutant lines and induced Q23 (FIG. 13B).

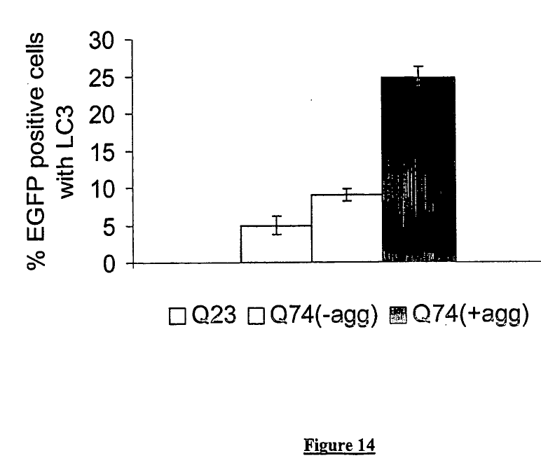

FIG. 14 shows a quantitative analysis of immunofluorescence data for EGFP-positive COS-7 cells expressing Q23 or Q74 with and without aggregates showing increased autophagic vacuoles (more than 15-20 vesicles per cell).

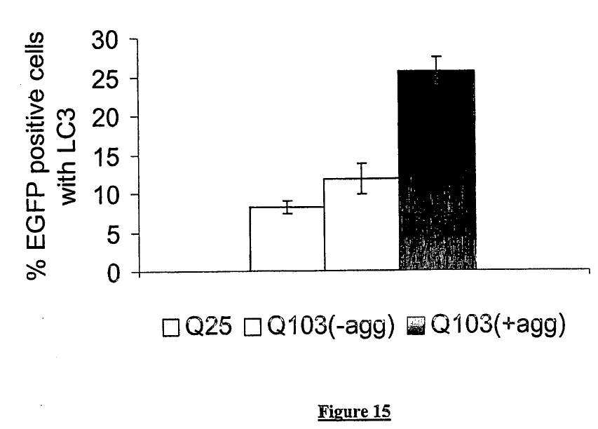

FIG. 15 shows a quantitative analysis of immunofluorescence data for COS-7 cells expressing Q25 or Q103 with anti-LC3 antibody showing increased autophagic vacuoles in Q103 expressing cells with aggregates. Data were analysed with ANOVA.

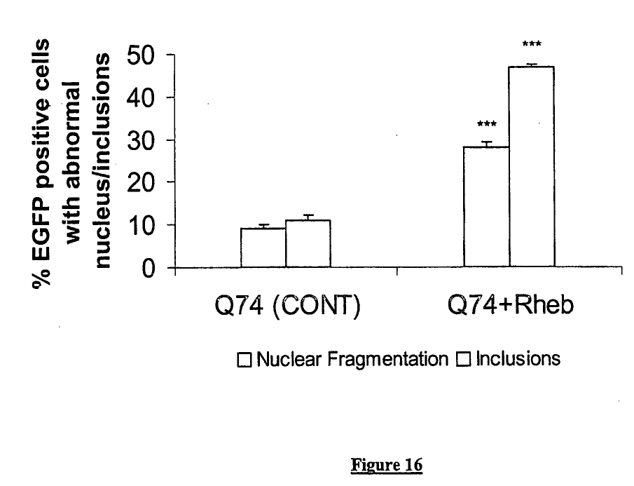

FIG. 16 shows an analysis of COS-7 cells cotransfected with mammalian expression vector encoding Rheb or empty vector control and Q74 for 48 hours. The percentages of EGFP-positive cells with aggregates or apoptotic nuclear morphology are dramatically increased with Rheb overexpression (Q74+Rheb).

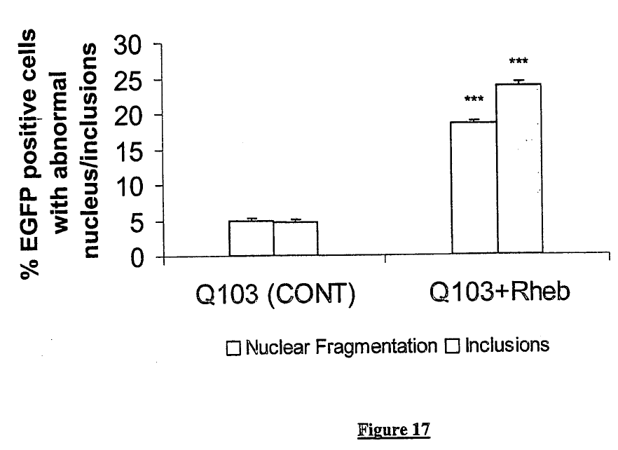

FIG. 17 shows an analysis of COS-7 cells cotransfected with mammalian expression vector encoding Rheb or empty vector control and Q103 for 48 hours. The percentages of EGFP-positive cells with aggregates or apoptotic nuclear morphology are dramatically increased with Rheb overexpression (Q103+Rheb).

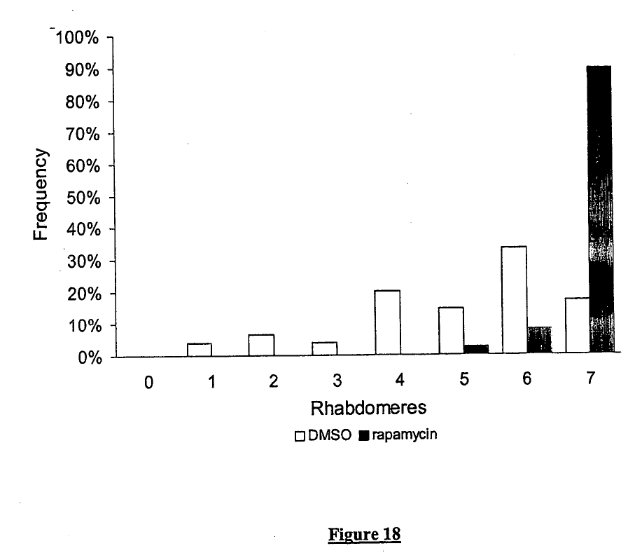

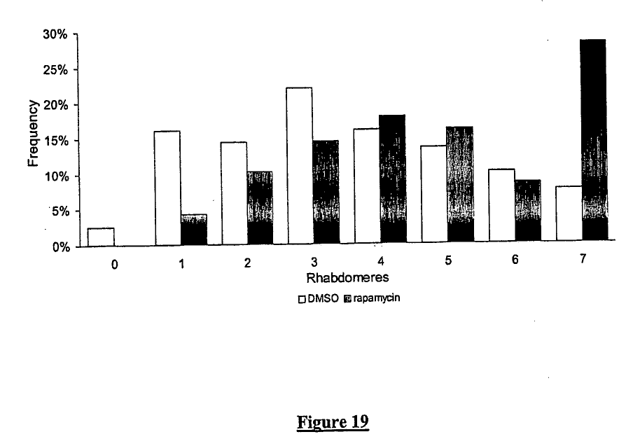

FIGS. 18 and 19 show a quantification of the effect of rapamycin on rhabdomeres at 2 days post-eclosion (FIG. 18) and at 3 days post-eclosion (FIG. 19), showing frequency of ommatidia with different numbers of visible rhabdomeres. Rapamycin treatment dramatically increases frequency of ommatidia with seven rhabdomeres in the HD 120Q flies. At both time points the effects is significant (p<0.0001; Mann-Whitney U test).

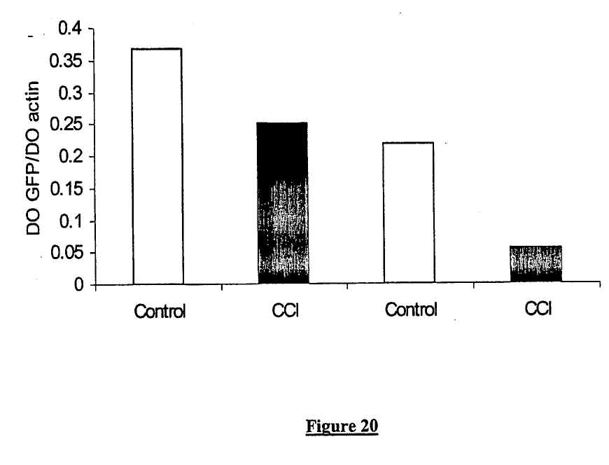

FIG. 20 shows a quantitation of data from a western blot of PC12 cells stably expressing huntingtin exon-1 fragment fused to GFP (Q74)m demonstrating decreased levels of soluble Q74 detected with an anti-GFP antibody after 48 h or 72 h of CCI-779 treatment. Quantification of huntingtin transgene as a function of actin (empty bars: control, black bars: CCI-779). This result was reproducible.

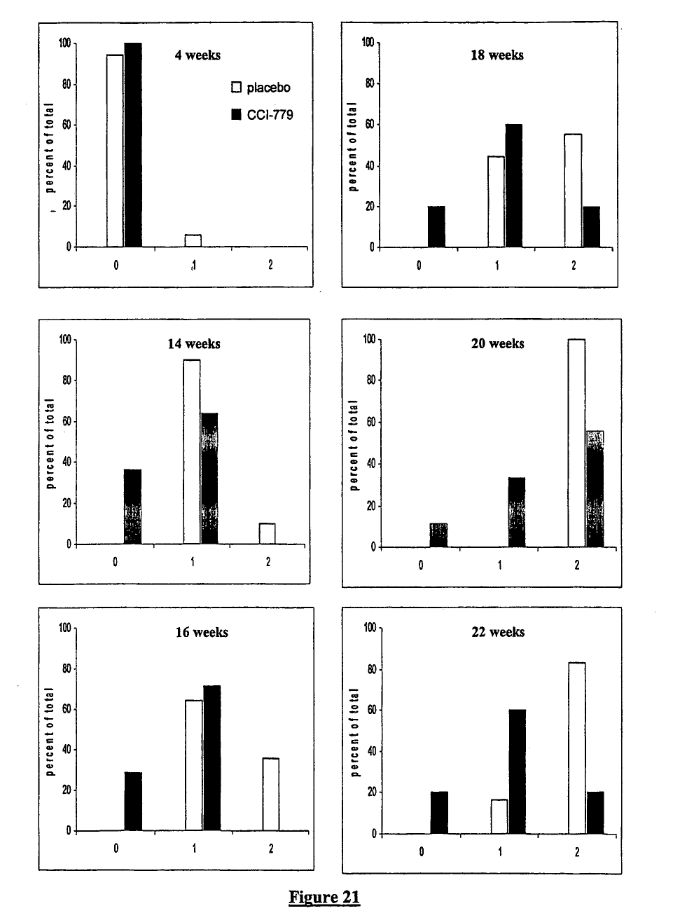

FIG. 21 shows that CCI-779 improves tremors in an HD mouse model expressing the N-terminal 1-171 of huntingtin with 82 glutamine repeats. 0=no tremors, 1=mild tremors, 2=marked tremors. Overall effect with data from all timepoints p=0.0006, Odds ratio=0.21 (95% confidence interval 0.098-0.45). p_{4 weeks}>0.77, p_{14 weeks}=0.20, p_{16 weeks}=0.01, p_{18 weeks}=0.08, p_{20 weeks}=0.14, p_{22 weeks}=0.07. Mice were tested aged 4 weeks (prior to treatment) (n_{CCI}=16, n_{placebo=}17), and after treatment at 14 weeks (n_{CCI}=11, n_{placebo}=10), 16 weeks (n_{CCI}=14, n_{placebo}=14), 18 weeks (n_{CCI}=10, n_{placebo}=9), 20 weeks (n_{CCI}=9, n_{placebo}=7), 22 weeks (n_{CCI}=5, n_{placebo}=6). Data from all timepoints assayed after starting treatment were assessed using a logistic regression approach. The control and treated groups were compared for each specific timepoint using Mann-Whitney U tests.

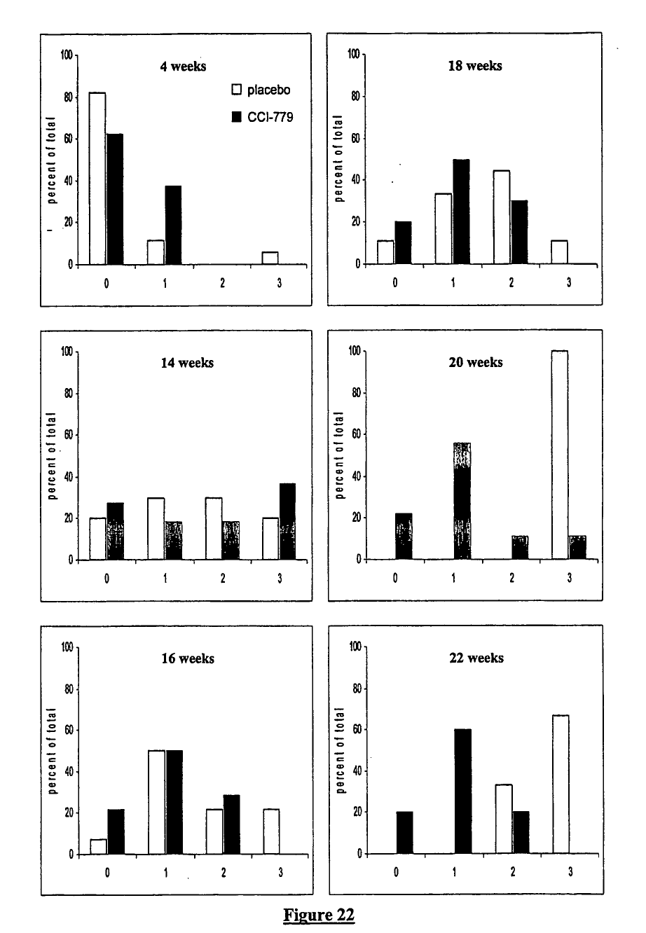

FIG. 22 shows the effect of CCI-779 on wire manoeuvre in an HD mouse model expressing the N-terminal 1-171 of huntingtin with 82 glutamine repeats. 0=active grip with hind legs; 1=difficulty grasping with hind legs; 2=unable to grasp with hind legs; 3=unable to lift hind legs, falls within 10 seconds; 4=falls immediately. Mice were tested aged 4 weeks (prior to treatment) (n_{CCI}=16, n_{placebo}=17), and after treatment at 14 weeks (n_{CCI}=11, n_{placebo}=10), 16 weeks (n_{CCI}=14, n_{placebo}=14), 18 weeks (n_{CCI}=10, n_{placebo}=9), 20 weeks (n_{CCI}=9, n_{placebo}=7), 22 weeks (n_{CCI}=5 n_{placebo}=6). Overall effect with data from all timepoints p=0.02 Odds ratio=0.51 (0.29-0.92); p4 _{weeks}=0.39, p_{14 weeks}=0.55, p_{16 weeks}=0.29, p_{18 weeks}=0.40, p_{20 weeks}=0.003, p_{22 weeks}=0.008.

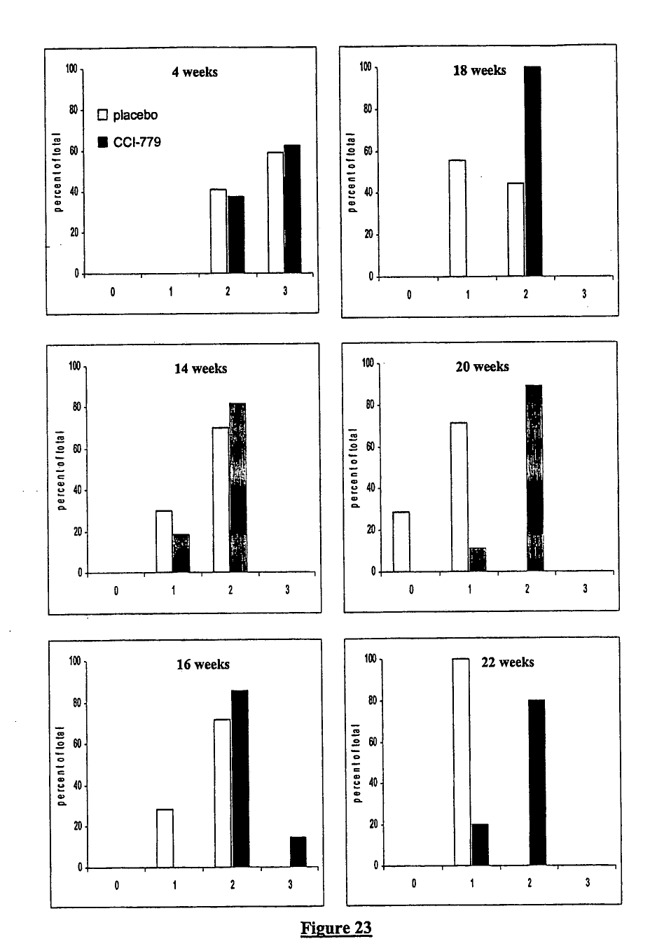

FIG. 23 shows the effect of CCI-779 on grip strength in an HD mouse model expressing the N-terminal 1-171 of huntingtin with 82 glutamine repeats. 0=no grip strength; 1=slight grip, semi-effective; 2=moderate grip, effective; 3=active grip, effective. Overall effect with data from all timepoints p<0.0001, Odds ratio=12.73 (4.35-37.2); p_{4 weeks}=0.85, p_{14 weeks}=0.6472, p_{16 weeks}=0.1827, p_{18 weeks}=0.0412, p_{20 weeks}=0.0021, p_{22 weeks}=0. 0285.

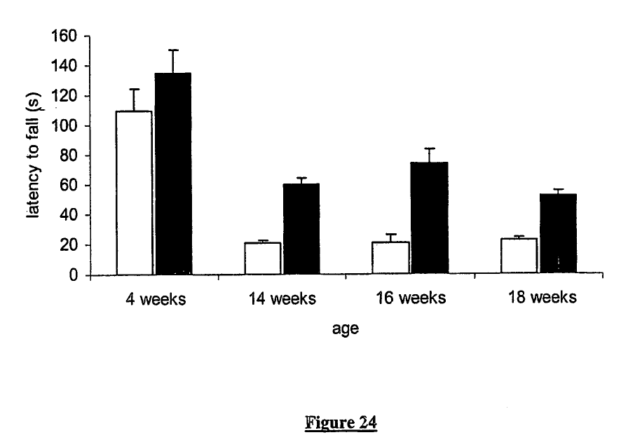

FIG. 24 shows accelerating rotarod performances in HD transgenic-mice expressing the N-terminal 1-171 of huntingtin with 82 glutamine repeats aged 4 weeks (before treatment) 14 weeks, 16 weeks, and 18 weeks, showing the difference in latency to fall from the rod between the placebo and CCI-treated groups. Scores between the control and treated groups were compared using a repeated measure ANOVA approach. Overall effect of CCI-779 using data from all treated timepoints p=0.035, p_{14 weeks}=0.012 (n_{CCI}=11, n_{placebo}=10), p_{16 weeks}=0.0025 (n_{CCI}=14, n_{placebo}=14), p_{18 weeks}=0.057 (n_{CCI}=10, n_{placebo}=9). p_{4 weeks}=0.227. CCI-779 had no discernable effects on the performance of non-transgenic animals on any of these behavioural tests.

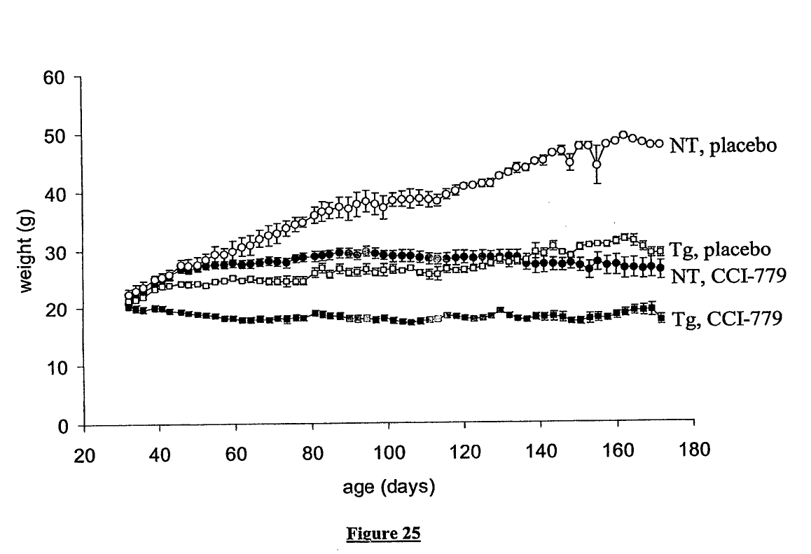

FIG. 25 shows the weights of HD-transgenic mice and non transgenic littermates treated with CCI-779 or placebo. Black squares: transgenic mice, CCI-779 treated; open squares: non-transgenic littermates, CCI-779 treated; black circles: transgenic mice, placebo-treated; open circles: non-transgenic littermates, placebo-treated.

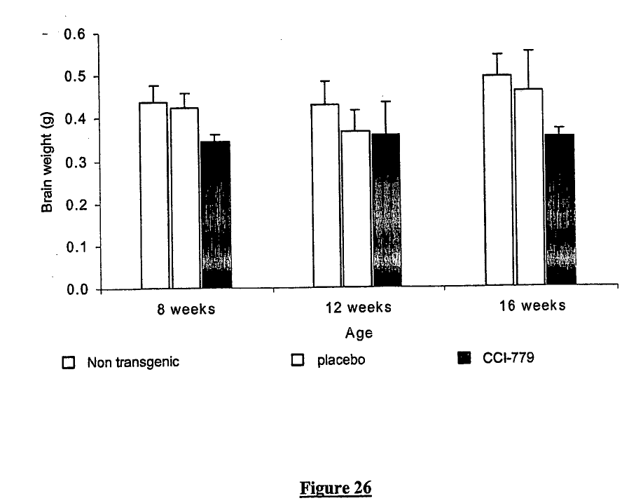

FIG. 26 shows the brain weights of HD transgenic mice treated with CCI-779 (black bars) or placebo (white bars) and non transgenic non treated littermates (grey bars) at 8 weeks old (n_{CCI}=3, n_{placebo}=3, n_{non transgenic}=7) (CCI vs. placebo p=0.023), 12 weeks old (n_{CCI}=2, n_{placebo}=3, n_{non transgenic}=5) (CCI vs placebo p=0.90), and 16 weeks old (n_{CCI}=2, n_{placebo}=3, n_{non transgenic}=2) (CCI vs pacebo p=0.22).

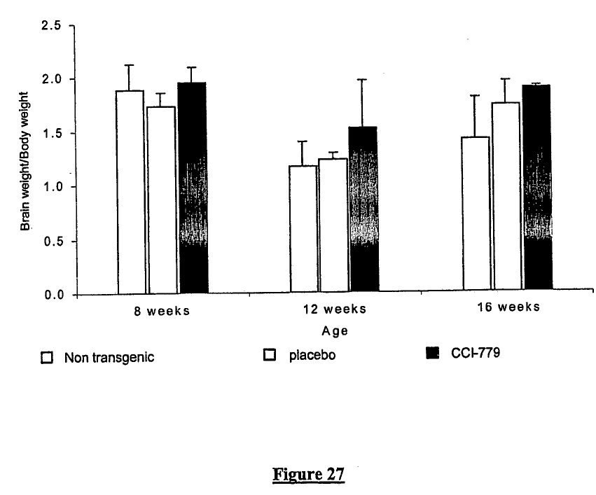

FIG. 27 shows brain weight/body weight of the HD transgenic mice treated with CCI-779 (black bars) or placebo (white bars) and non-transgenic non-treated littermates (grey bars) at 8 weeks old (n_{CCI}=3, n_{placebo}=3, n_{non transgenic}=7) (CCI vs placebo p=0.06), 12 weeks old (n_{CCI}=2, n_{placebo}=3, n_{non transgenic}=5) (CCI vs placebo p=0.38), and 16 weeks old (n_{CCI}=2, n_{placebo}=3, n_{non transgenic}=2) (CCI vs placebo p=0.56).

### EXPERIMENTAL

Materials and Methods

Plasmids

Mammalian expression vectors comprising EGFP (pEGFP-C1, Clontech) fused at its C-terminus with a Huntington's Disease gene exon 1 fragment with 74 polyglutamine repeats (Q74) or a polyalanine stretch of 19 repeats (A19) were used (Rankin, J. et al (2000) Biochem. J., 348, 15-19; Narain, Y. et al (1999) J. Med. Genet., 36, 739-746.). A Haemagglutinin-tagged Huntington's Disease gene exon 1 fragment with 74 polyglutamine repeats in pHM6 vector (Q74-HA) was also used. The PC12 stable lines expressing exon 1 of Huntington gene were as described in Wyttenbach A. et al., (2001) Hum. Mol. Genet. 10 1829-45.

Mammalian Cell Culture and Transfection

African green monkey kidney cells (COS-7) were grown in Dulbecco's Modified Eagle Medium (DMEM, Sigma) supplemented with 10% Fetal Bovine Serum (FBS), 100 U/ml Penicillin/Streptomycin, 2 mM L-Glutamine and 1 mM Sodium Pyruvate at 37° C., 5% Carbon dioxide. The cells were grown on coverslips in six-well plates for immunofluorescence analysis, or were directly grown in six-well plates to 60-80% confluency for 24 hours for western blot analysis. Transfection was done using LipofectAMINE reagent (Invitrogen) using the manufacturer's protocol. The transfection mixture was replaced by normal culture medium after 5 hours incubation at 37° C. and the transfected cells were analysed by immunofluorescence or immunoblot 48 hours after transfection. The cells were left untreated or treated with 10 mM 3-methyl adenine (3-MA, Sigma), 0.5 mM N⁶, N⁶, Dimethyl adenosine (DMA, Sigma), 0.2 μg/ml Rapamycin (Sigma), 200 nM Bafilomycin A1 (BafA1, Sigma), 10 μM Lactacystin (Sigma) or 10 μM epoxomicin (Affinity research products ltd.) for 15 hours before fixation for immunofluorescence or processing for western blots. DMA, BafA1, epoxomicin and rapamycin were dissolved in DMSO and 3-MA and lactacystin were in water. Equal amounts of water or DMSO were added to the untreated controls, where relevant. The cells on coverslips were rinsed with 1×PBS, fixed with 4% paraformaldehyde in 1×PBS for 20 min and mounted in antifadent supplemented with 4′,6-diamidino-2-phenylindole (DAPI, 3 μg/ml, Sigma) to allow visualisation of nuclear morphology. The PC12 stable cells were maintained at 75 μg/ml hygromycin in standard medium consisting of high glucose DMEM (Sigma) with 100 U/ml penicillin/streptomycin, 2 mM L-glutamine (Invitrogen), 10% heat-inactivated horse serum (Invitrogen), 5% Tet-approved fetal bovine serum (FBS) (Clontech) and 100 μg/ml G418 (Invitrogen) at 37° C., 10% CO2. The cells were seeded at 1-2×10⁵ per well in 6-well plates and were induced with 1 μg/ml doxycycline (Sigma) for 8 hours. The expression of transgenes was switched off by removing doxycycline from the medium. Cells were either left untreated or treated with 3-MA, BafA1, epoxomicin, lactacystin, rapamycin, 10 μg/ml cycloheximide (Sigma) or cycloheximide+rapamycin at the concentrations specified above for 24, 48 or 72 hours and the medium with the inhibitors/activator changed every 24 hours. The cells were then scraped off from the wells into 1.5 ml eppendorf tubes, pelleted at 8000 rpm and washed twice with 1×PBS. They were either fixed with 4% paraformaldehyde for 20 minutes and mounted in DAPI over coverslips on glass slides or processed for western blot analysis. CCI-779 was supplied by Wyeth Pharmaceuticals (Philadelphia). For tissue culture experiments, a stock solution of 1M in ethanol was prepared the day of experiment and was diluted in the appropriate medium

Western Blot Analysis of PolyQ and PolyA Expression Products

The pellets for westerns from COS-7 or PC12 cells were lysed on ice in Laemmli buffer (62.5 mM Tris-HCl pH6.8, 5% β-mercaptoethanol, 10% glycerol, and 0.01% bromophenol blue) for 30 min in the presence of protease inhibitors (Roche Diagnostics). The lysates were subjected to SDS-PAGE (10%) electrophoresis and proteins transferred onto nitrocellulose membrane (Amersham Pharmacia Biotech). The primary antibodies used include mouse monoclonal anti-GFP antibody (8362-1, Clontech) at 1:2000, rabbit monoclonal anti-actin antibody (A2066, Sigma) at 1:3000, mouse monoclonal anti-tubulin antibody (Clone DM 1A, Sigma) at 1:1000 and mouse monoclonal anti-HA antibody (Covance) at 1:1000. Blots were probed with Horse Radish Peroxidase (HRP) conjugated anti-mouse or anti-rabbit IgG (Bio Rad) at 1:2000. Bands were visualised using the ECL detection reagent (Amersham).

Quantification of Aggregate Formation and Abnormal Cell Nuclei

Aggregate formation and nuclear morphology were assessed using a fluorescence microscope. 200 EGFP positive COS-7 cells were selected and the proportion of cells with aggregates was counted. Aggregates are described in Rankin et al (2000) and Narain et al (1999) supra. Cells were considered dead if the DAPI-stained nuclei showed apoptotic morphology (fragmentation or pyknosis). Pyknotic nuclei are typically <50% diameter of normal nuclei and show obvious increased DAPI intensity. These criteria are specific for cell death, as they show a very high correlation with propidium iodide staining in live cells). Furthermore, these nuclear abnormalities are reversed with caspase inhibitors (Wyttenbach, A. et al (2000) Proc. Natl. Acad. Sci. USA, 97, 2898-2903). Analysis was performed with the observer blinded to the identity of the slides and all experiments reported in the figures were done in triplicate at least twice.

Statistical Analysis

The P-values were determined by unconditional logistical regression analysis using the general loglinear analysis option of SPSS Ver. 6.1. software (SPSS, Chicago, USA).

Establishment of Inducible α-synuclein Expressing PC12 Cell Lines

Human α-synuclein with a HA tag at the N-terminus and a 6His tag at the C-terminus was amplified from pHM6α-synuclein constructs (Furlong, R. A. et a; 1 (2000) Biochem. J. 346, 577581) and inserted into pTRE2 hyg vector (Clontech) using Nhe I/Sal I sites. Constructs were confirmed by sequencing before use. The pTRE2 hygα-synuclein construct and pTettTS (Clontech) were cotransfected into PC12 TetOn cells (Clontech), using Lipofectamine (Invitrogen) and Plus Reagent (Invitrogen). Single colonies were isolated using cloning cylinders (Sigma) and cells were grown and tested for α-synuclein expression upon induction. Cell lines were subjected to a further round of purification from single colonies to ensure that lines were pure.

Cell Culture

PC12 cells were grown in Dulbecco's modified eagle's medium (DMEM, Sigma) supplemented with 10% horse serum (Sigma), 5% fetal bovine serum (Sigma), 100 U/ml penicillin/streptomycin, 2 mM L-glutamine, 50 mg/ml G418 (Invitrogen) and 149 mg/ml hygromycin B (Calbiochem) at 37° C., 10% CO₂. To induce differentiation, cells were grown in media containing 1% horse serum and 100 ng/ml nerve growth factor (2.5S, Upstate Biotechnology) and incubated for about 5 days. Cells were induced to express synuclein with 2 mg/ml doxycycline (Sigma).

Immunofluorescence

Coverslips were placed in 6 well dishes and coated with 0.01% poly-L-lysine (Sigma). Cells were seeded and induced with 2 mg/ml doxycycline and incubated as necessary. Cells were fixed with 4% paraformaldehyde (Sigma) for 30 minutes, then washed with-PBS and permeabilised with 0.1% Triton X100 (Sigma) for 15 minutes. Cells were blocked in 10% fetal calf serum for at least 30 minutes. Anti α-synuclein monoclonal antibody (BD Biosciences) was used at 1:200 for 2-16 hours, cells were washed and 1:200 Cy3-conjugated antimouse antibody (Jackson ImmunoResearch labs) was added for 1 hour. This anti α-synuclein antibody could detect both human and rat α-synuclein, therefore uninduced controls were performed in parallel to compare endogenous α-synuclein levels. Staining was always considerably fainter in the uninduced controls. For double staining with LysoTracker Red (Molecular Probes) cells were incubated in Earle's Balanced Salts Solution (Sigma) with 75 nM LysoTracker for 2 hours at 37° C. Cells were fixed and stained with anti α-synuclein antibody and 1:200 FITC conjugated antimouse antibody (Jackson ImmunoResearch labs) was used as secondary antibody. Slides were mounted in Citifluor (Citifluor Ltd) with 3 μg/ml 4′,6′Diamidino-2-phenylindole (DAPI, Sigma). Cells were visualised using a Zeiss LSM510 confocal microscope.

Treatment with Autophagy/Proteasome Drugs

Cells were induced for 24 hours and then washed twice with media to remove doxycycline. Then cells were incubated in 25 media containing either 10 mM 3-methyladenine (3MA, Sigma), 200 nM bafilomycin A1 (Sigma), 0.2 mg/ml rapamycin (Sigma), 10 mM lactacystin (Sigma), 10 mM epoxomicin (Affinity Research Products Ltd) or carrier controls (water or DMSO (Sigma)). 3MA and lactacystin were made up in water and rapamycin, epoxomicin and bafilomycin were dissolved in DMSO.

After 24 hours the media was replaced with fresh media plus drug and after a further 24 hours cell pellets were collected and stored at −80° C. until required.

Western blot analysis of α-Synuclein Expression

Cell pellets were collected and stored at −80° C. until needed. Cells were lysed on ice in lysis buffer: 1% TritonX100, 20 mM TRIS pH7.5, 137 mM NaCl, 1 mM EGTA, 10% glycerol, 1.5 mM MgCl2 and protease inhibitor cocktail (Complete, Roche).

Samples were then mixed with loading buffer: 62.5 mM TRIS pH 6.8, 2% SDS, 10% glycerol, 0.05% bromophenol blue, 100 mM dithiothreitol and 700 mM β-mercaptoethanol and boiled before loading onto 14% denaturing polyacrylamide gels.

Each lane was loaded with protein from a similar number of cells, based on cell counting at the time of seeding. Proteins were transferred onto Hybond ECL nitrocellulose membrane (Amersham). α-Synuclein was detected with an antiHA monoclonal antibody (Covance) at 1:1000 dilution with 4-18 hours incubation. HRP conjugated antimouse antibody (Amersham) at 1:2000 dilution was then added to blots and antibody was detected using ECL blotting reagents (Amersham) and Hyperfilm ECL (Amersham). Blots were stripped and then reprobed for α-tubulin (Sigma) at 1:1000 dilution.

Western Blot Analysis of Mouse Tissue

Western blot analysis was done using standard techniques with ECL or ECL Plus detection kit (Amersham). The primary antibodies used include anti-GFP (Clontech), anti-mTOR, anti-Phospho-mTOR (Ser 2448), anti-p70 S6 Kinase, anti-Phospho-p70 S6 Kinase (Thr389) 1A5, anti-4E-BP1, anti-phospho-4E-BP1 (Thr37) all from Cell Signaling technology, anti-huntingtin (EM48, Chemicon) and anti-tubulin (Sigma). Brains from nine HD transgenic mice (N-terminal 171 amino acids with 82 glutamine repeats) ¹⁶ and age-matched wild-type littermate controls were used. 2.5 volume of buffer B (50 mM Tris pH 7.5, 10% glycerol, 5 mM magnesium acetate, 0.2 mM EDTA, 0.5 mM DTT and protease inhibitor) was added to sliced brain tissue and homogenised at 4° C. The homogenate was spun at 12,000 rpm at 4° C. The supernatant was removed and used for western blot analysis.

For immunoprecipitation, cells were lysed in RIPA buffer (1×PBS, 1% Nonidet P-40, 0.5% sodium deoxycholate, 0.1% SDS) and immunoprecipiated using relevant antibodies.

Immunogold Labelling for Electron Microscopy

Cells were induced with 2 mg/ml doxycycline for 48 hours. The cells were fixed in situ with 2% formaldehyde and 0.05% glutaraldehyde in 0.1M PIPES buffer and harvested by scraping. The cells were incubated in 0.1M PIPES buffer containing 5% BSA and 20% polypropylene glycol, concentrated by centrifugation at 2000 rpm in a Hermle Z 160 M centrifuge (Hermle Labortechnik) and the supernatant was removed.

Small droplets (5 ml) of the cells were mounted onto aluminium foil and quench frozen, by plunging them into melting propane cooled in liquid nitrogen. After freezing, the cells were transferred into a Leica AFS freeze substitution unit in vials of frozen, dry methanol, containing 0.5% uranyl acetate. They were maintained at −90° C. for 24 hours followed by 24 hours at −70° C. and another 24 hours at −50° C. They were infiltrated with Lowicryl HM20 over 3 days and polymerised by irradiation with UV light for 48 hours. Thin sections were cut using a Leica Ultracut S and mounted on Formvar coated nickel grids. The sections were incubated overnight in mouse AntiHA primary antibodies (Covance), diluted 1:5 in Tris Buffered Saline (TBS) at pH 7.4 containing 0.1% Tween 20, 0.1% Triton X100, 0.5% fetal calf serum and 10% normal goat serum. The sections were washed six times in TBS and incubated with goat antimouse immunoglobulins conjugated to 10 nm gold particles (British Biocell), diluted 1:100 in the diluent for the primary antibody at pH 8.5 without added goat serum for 1 hour (25). They were rinsed 6 times in TBS and twice in deionised water and stained with uranyl acetate and lead citrate before viewing in a Philips CM100 transmission electron microscope.

Immunocytochemistry/Immunohistochemistry

Immunocytochemistry was done in COS-7 cells fixed with 4% paraformaldehyde (Sigma). Free-floating mouse brain sections from HD transgenic and control brains and paraffin-embedded HD grade III and control brain slices spanning the caudate and putamen regions were used for histochemical analysis. Primary antibodies used: anti-mTOR, anti-phospho-mTOR (Ser2448), anti-4E-BP1, anti-phospho-S6 (Ser235/236) (Cell signalling technologies), anti-LC3 (kind gift from Dr Yoshimori) and anti-huntingtin and anti-ubiquitin (both from Chemicon). Immunohistochemistry was performed by standard fluorescence methods or peroxidase labelling using Vectastain Avidin: Biotinylated enzyme Complex (ABC) kit. Relevant negative controls without the primary antibodies were performed alongside all the experiments.

Luciferase Assay

PC12 cells stably expressing Q23 or Q74 were transfected with a luciferase construct having a 5′ untranslated region from the eEF2 gene containing a terminal oligopyrimidine tract (TOP-luciferase) or without the TOP sequence (Control-luciferase) along with β-galactosidase (to control transfection efficiency). Transfected cells were maintained in serum free medium with doxycycline for 36 hours following which the cells were stimulated with 15% serum with or without rapamycin for 3 h. Cells were then lysed and luciferase assays were performed according to standard protocol. COS-7 cells were co-transfected with Q23 or Q74 with control- or TOP-luciferase along with β-gal and experiments were performed as above. In addition to the classical TOP construct, we have also used a construct with a non-classical TOP sequence that shows rapamycin-sensitivity and appropriate control construct (Kim & Chen (2000) supra). As both control and TOP luciferase constructs have the same promoters and transfection is controlled for with β-gal construct, the rapamycin-sensitive reduction in luciferase activity that is specific to the TOP construct has been considered to be an indication of the dependency of the TOP sequences on mTOR-regulated translational ability (Schwab, M. S. et al. Mol Cell Biol 19, 2485-2494 (1999)). Experiments were performed in sextuplicate and repeated several times.

Rapamycin Treatment of Drosophila:

A Drosophila line y; w; gmr-Q120 was crossed to w; iso2; iso3 to obtain progeny heterozygous for the transgene gmr-Q120. Flies were allowed to mate on normal food for 2-3 days and then transferred to food containing 1 μM rapamycin (Sigma) or DMSO. After eclosion, flies were placed on food containing 1 μM rapamycin or DMSO. Flies were transferred to newly prepared food every day.

Pseudopupil analysis: Adult male flies were decapitated and the head was mounted onto a microscope slide using nail polish. Analysis was performed with an optical microscope using a 60× objective with the observer blinded to the identity of the slides. Approximately 75 ommatidia were evaluated for each group (approx. 15 ommatidia in 5 individuals). Each experiment was done twice. The frequency of ommatidia with different numbers of visible rhabdomeres was based on all visible photoreceptor neurons, irrespective of their shape or brightness. Flies were raised at 25° C. with 12/12-h light/dark-cycle, 70% humidity.

Mice and Behavioural Tests

All studies and procedures were performed under the jurisdiction of appropriate Home Office Project and Personal animal licences and with local ethical committee approval. HD-N171-N82Q mice²³ expressing the first 171 amino acids of the human huntingtin under the expression of a mouse PrP promoter were genotyped at 3 weeks of age by PCR using the following set of primers:

| | |
|---|---|
| IL-2F: | (CTAGGCCACAGAATTGAAAGATCT), |
| IL-2R: | (GTAGGTGGAAATTCTAGCATCATCC), |
| HD82Q-F: | (GTGGATACCCCCTCCCCCAGCCTAGACC), |
| HD591-5′R: | (GAACTTTCAGCTACCAAGAAAGACCGTGT). |

CCI-779 was prepared in ethanol as a stock solution at 50 mg·ml⁻¹, the day of experiment and diluted at 2 mg·ml⁻¹ in 0.15M NaCl, 5% Tween 20, 5% PEG 400 immediately before injection. Transgenic mice and non-transgenic littermates were compared in the trial. There were no significant differences in test performances in the mice assigned to the treatment and placebo groups at 4 weeks and no differences in the sex ratios in the groups. Mice were weighed three times a week, then injected intra peritoneally with a 1% v/w solution of CCI-779 or placebo, from 4 weeks old to 16 weeks of age and then every other week until 21 weeks of age. Mice were monitored daily. Motor performances were assessed at 4, 14, 16 and 18 weeks of age with a rotarod apparatus (Accelerating Model, Ugo Basile, Biological Research Apparatus, Varese, Italy)—the licence under which these experiments were performed limited rotarod assessments to four testing time points. The mice were given three training sessions per day, for two consecutive days, to acclimatise them to the apparatus. On the third day the mice were given six separate trials. On the training and testing days the speed was set to increase from 4 to 40 rpm in 250 sec; the animals were put on the rotarod for a maximum of 300 sec. Note that mice were not injected in the weeks when they were tested for rotarod performance, in order to avoid any confounding effects of the injections. Grip strength, wire manoeuvre and tremor monitoring are part of the SHIRPA battery of behavioural tests³⁰. These tests were assessed at 4, 14, 16, 18, 20 and 22 weeks of age. For grip strength of the forelimbs, the mice were lifted by the tail, allowed to hold a metal grid, and gently pulled horizontally backward until they could no longer hold the grid. They were scored as follows: 0=none; 1=slight grip, semi-effective; 2=moderate grip, effective; 3=active grip, effective.

For tremors, the mice were placed on a grid in a clear perspex cylinder for 5 minutes. The tremors were recorded for the last 2 minutes and the mice received a mark as follows: 0=none; 1=mild; 2=marked.

For the wire manoeuvre, the animals were held above a horizontal wire by tail suspension and lowered to allow the forelimbs to grip the wire. The mice were held in extension, rotated around to the horizontal and released. They received a mark as follows: 0=active grip with hind legs; 1=difficulty grasping with hind legs; 2=unable to grasp with hind legs; 3=unable to lift hind legs, falls within 10 seconds; 4=falls immediately.

Statistics

P-values were determined with t tests, repeated measure or factorial ANOVA, where appropriate, for parametric data and with Mann-Whitney U tests for non-parametric data, using the STATVIEW software, version 4.53 (Abacus Concepts, Berkeley, Calif.). For mouse behavioural data that were non-parametric and analysed at multiple time points (tremors, grip strength and wire manoeuvre), we used the following strategy to provide a measure of the significance of the pooled data across all the treated time points. Scores between the treated and control groups were compared using a logistic regression approach. Effect size was assessed in terms of the odds ratio per unit change in score. To allow for the fact that the same mice were observed repeatedly, significance levels were assessed by simulation. To do this, the treatment groups were randomly permuted among the mice 10,000 times, and the significance level was determined as the proportion of simulations for which the Z-score for the treatment effect from the logistic regression exceeded the observed value. 95% confidence limits for the odds ratio were obtained by using the robust variance approach. All calculations were performed in Stata.

Results

Inhibition of the Sequestration Stage of Autophagy Increases Aggregate Formation and Cell Death in COS-7 Cells Expressing Mutated HD Exon 1 or Polyalanine Protein

An exon 1 fragment of the Huntington's disease (HD) gene with 74 or 55 glutamines (Q74/Q55) fused to enhanced green fluorescent protein (EGFP) was analysed. The effects of perturbing autophagy on the HD constructs were compared with another aggregate-prone protein—EGFP fused to 19 alanine repeats (A19). Both the Q74/55 and A19 constructs form aggregates and are associated with increased cell death, compared to Q23 and A7 constructs or empty EGFP vectors, which do not form any aggregates under the same expression conditions (Wyttenbach, A. et al (2000) Proc. Natl. Acad. Sci. USA, 97, 2898-2903; Rankin, J. et al (2000) Biochem. J. 348, 15-19).

The role of autophagy in degrading these proteins was examined using the specific inhibitor 3-methyl adenine (3-MA) (Kovács, A. L et al (1998) Biol. Chem. 379, 1341-1347). 3-MA inhibits autophagy at the sequestration stage, where a double membrane structure forms around a portion of the cytosol and sequesters it from the rest of cytoplasm to form the autophagosome (Klionsky, D. J. and Oshumi, Y. (1999) Annu. Rev. Cell Dev. Biol. 15 1-32). 3-MA treatment resulted in an obvious change in the appearance of the aggregates formed by Q74 or A19 constructs transiently transfected into COS-7 cells. In cells with aggregates, 3-MA increased their apparent size and number. 3-MA treatment also increased the proportions of Q74- or A19-expressing cells with aggregates and this was accompanied by an increase in cell death (FIG. 1). 3-MA did not cause aggregate formation in COS-7 cells-expressing EGFP tagged wild-type HD exon 1 protein with 23 glutamine repeats or an EGFP-polyalanine protein with 7 repeats. Identical aggregate phenotypes and similar significant increases in the proportions of Q74- and A19-transfected cells with aggregates or cell death were observed after treatment with another inhibitor of the sequestration stage of autophagy, N⁶,N⁶-dimethyladenosine (DMA). Similar results were obtained with Q55 and Q74). The increased aggregation of Q74 and A19 caused by 3-MA was associated with an increase in the levels of the transgene protein (about 80% transfection efficiency was obtained in COS-7 cells). 3-MA treatment did not cause any change in the levels of empty EGFP transfected in the same way into COS-7 cells. However it did cause an increase in the levels of an HA-tagged version of the same HD exon 1 fragment with Q74. Thus, the 3-MA is not acting simply on the EGFP part of the fusion proteins we have studied. These results provide indication that inhibiting the sequestration stage of autophagy results in increased levels of the mutant proteins, thereby enhancing their aggregation.

Inhibition of Autophagosome-Lysosome Fusion Increases Aggregation of Q74 or A19 in COS-7 Cells

After the sequestration step, the autophagosome needs to fuse with the lysosome in order for its contents to be degraded. This next step was tested with the vacuolar ATPase inhibitor Bafilomycin A1 (BafA1), which interferes with the autophagosome-lysosome fusion step, possibly because lysosomal acidification is required for this fusion (Yamamoto, A. et al (1998) Cell Struct. Funct. 23, 33-42). As seen in FIG. 2, treatment with BafA1 resulted in a change in aggregate morphology (increased size) and increased the proportions of transfected Q74 or A19 COS-7 cells with aggregates similar to the results with 3-MA. This was also accompanied by an increase in cell death in these cells.

3-MA and BafA1 Decrease Turnover of Q74 Aggregates in Stable Inducible PC12 Cells

A stable doxycycline-inducible PC12 cell line expressing EGFP-tagged HD exon 1 with Q74 (Wyttenbach, A. et al. (2001) Hum. Mol. Genet. 10, 1829-45) was used to allow transgene expression to be specifically switch off by removing doxycycline from the medium, without interfering with ongoing cellular protein synthesis. This is important because autophagy is protein-synthesis dependent (Lawrence, B. P. and Brown, W. J. (1993) J. Cell Sci., 105, 473-480; Abeliovich, H. et al (2000) J. Cell Biol. 151, 1025-1034). The stable lines were induced for 8 hours (8 h ON) and expression was then switched off by removing doxycycline from the medium for the next 24, 48 or 72 hours, which we have called 24, 48 or 72 h OFF. The proportion of cells with aggregates peaked at 24 h OFF and subsequently was reduced over time. The peak at 24 h OFF may reflect a delay in the wash-out of doxycycline, or be a function of the kinetics of aggregation—aggregation occurs after a relatively long lag phase. Western blot analysis of these cells shows aggregates as a high molecular weight band in the stack at 48 h OFF but these have largely disappeared at 72 h OFF. The reduction in aggregates from 24 to 48 to 72 hours correlated with a reduction in the amounts of soluble protein. In order to test the effects of 3-MA or BafA1 on the turnover of aggregates, the cells were induced for 8 hours, then switched off for 48 or 72 hours with these treatments. Treatment with either of the inhibitors (72 h OFF+3-MA and 72 h OFF+BafA1) resulted in increased numbers of aggregate containing cells when compared to time-matched control (72 h OFF). This is also supported by the results of the western blot—which showed that 3-MA or BafA1 resulted in increased aggregate formation, as seen by the high molecular weight fraction on the stack, which persists even 72 hours after switching off the expression (compared with time-matched untreated controls). These results provide indication that inhibiting the autophagy-lysosomal pathway interferes with the degradation of the mutant HD exon 1 protein/aggregates, which is consistent with the transient transfection experiments in COS-7 cells.

Induction of Autophagy Decreases Aggregation and Cell Death in Mammalian Cells Expressing Q74 and A19

The effect of inducing autophagy with an antifungal macrolide antibiotic, rapamycin, was examined. Treatment with rapamycin resulted in a decrease in the proportions of aggregate-containing cells and cell death in COS-7 cells expressing A19 but no significant change was observed in cells expressing Q74 after 48 hours of transfection (FIG. 3A). The aggregates formed by Q74 at 48 h were much larger than at 24 hours and the aggregation rate in the untreated Q74 cells was higher when compared to the A19 aggregates (FIG. 3A). Thus, Q74 may aggregate more rapidly than A19 and more quickly form highly stable structures that are relatively resistant to rapamycin (autophagy). We therefore repeated the experiment at 24 hours post transfection with Q74, when fewer cells have formed aggregates (FIG. 3B). Under these conditions, rapamycin reduced the proportions of cells with aggregates and cell death (FIG. 3B). We performed similar experiments in the stable inducible PC12 cell line expressing EGFP-Q74. The cells were induced for 8 hours and then expression was switched off for 24, 48 or 72 hours, with or without rapamycin treatment. The rate at which protein clearance occurred was measured by the loss of fluorescence and also by comparing the protein levels on a western blot. Treatment with rapamycin resulted in a loss of fluorescence ((i) 72 h OFF+Rap vs. 72 h OFF) and also a decrease in protein levels on a western blot when compared to the untreated control. Since rapamycin is a slow inhibitor of protein synthesis, we checked whether inhibition of protein synthesis by treatment with cycloheximide had a similar effect on Q74 clearance and aggregation. In contrast to rapamycin, cycloheximide alone increased cell fluorescence and the number of visible aggregates (72 h OFF+Cycloheximide). Indeed, cycloheximide abrogated the effect of rapamycin on these cells (72 h OFF+Rap&Cycloheximide), consistent with the observation that cycloheximide causes a drastic reduction in autophagy-induced protein degradation (Abeliovich, H. et al (2000) J. Cell Biol. 151, 1025-1034).

Effect of Proteasome Inhibition by Epoxomicin

The effect of epoxomicin, a potent and selective proteasome inhibitor (Meng, L. et al (1999) Proc. Natl. Acad. Sci. USA, 96, 10403-10408) was tested in COS-7 cells expressing Q74 or A19 and in PC12 cells stably expressing Q74.

Immunofluorescence revealed a change in morphology of Q74 or A19 aggregates in COS-7 cells treated with epoxomicin. As described previously with Q74 and lactacystin (Wyttenbach, A. et al (2000) supra), there were more aggregates in each aggregate-containing cell. However, epoxomicin treatment did not affect the proportion of Q74 expressing cells with aggregates or apoptotic nuclear morphology. This is in contrast to lactacystin, which increases the proportion of Q74-expressing cells with aggregates. However, epoxomicin did increase the proportion of COS-7 cells expressing A19 with aggregates (FIG. 4). PC12 cells stably expressing Q74 were induced for 8 hours and expression was switched off for 48 hours without (48 h OFF) or with treatment with epoxomicin (48 h OFF+Epox) or lactacystin (48 h OFF+Lac). Surprisingly, epoxomicin resulted in a reduction in the number of aggregate-containing cells when compared to control, even though it caused an obvious increase in cellular fluorescence. This was confirmed by western blot analysis of epoxomicin-treated cells which revealed a marked increase in the soluble fraction but failed to detect any high molecular weight bands corresponding to aggregates. This is in contrast to lactacystin, which increased Q74 aggregation immunocytochemically (48 h OFF+Lac) and both the soluble and insoluble fractions on western blots. A marked increase was observed in the heat shock protein 70 on epoxomicin treatment, consistent with inhibition of the proteasome (Kim, D., Kim, S. H. and Li, G. C. (1999) Biochem. Biophys. Res. Com., 254, 264-268).

Establishment of Inducible α-Synuclein Cell Lines.

Stable, inducible lines were made for human wild-type, A30P and A53T α-synuclein in PC12 (rat phaeochromocytoma) cells using the TetOn system, where addition of doxycycline switches on transgene expression. Two different clonal lines were selected for each form of α-synuclein, on the basis of low background transgene expression and high inducibility.

α-Synuclein localisation was examined in the induced cells. α-synuclein was found to be evenly distributed across the cells, often with a vesicular pattern of staining. The diffuse cytoplasmic and nuclear localisation of α-synuclein was similar to previous observation with this protein in PC12 cells (Rideout, H. J. et al (2001) J. Neurochem. 78, 899908). At the light microscope level, it was unclear if the vesicular structures were aggregates. We did not see very large aggregates characteristic of polyglutamine and polyalanine expansions for either wildtype, A30P or A53T, even after expression of α-synuclein for 10 days. The staining for α-synuclein in uninduced cells was below the level of detection when analysed with confocal microscopy using the same settings that gave clear signals for induced cells. However, α-synuclein immunoreactivity in the uninduced cells was observed when the gain was increased—this staining is likely to reflect predominantly endogenous α-synuclein in the cells, since there is minimal leakiness of transgene expression in uninduced cells.

Cell death was examined by FACS analysis and inspection of nuclear morphology after DAPI staining. No significant change in numbers of dead cells was observed after induction of α-synuclein expression in any of the lines expressing wildtype, A30P or A53T. In both cycling and differentiated cells, cell death was <10% at all times studied up to 10 days of induction (assessment by DAPI staining for wildtype, A30P or A53T). α-Synuclein levels were determined by western blotting using an antiHA antibody to enable specific detection of the transgene products. All blots were performed at least twice with each clonal cell line. Expression of α-synuclein was switched on with doxycycline and then switch off expression by removing doxycycline from the medium, in order to follow α-synuclein degradation. Significantly lower levels of α-synuclein were observed 72 hours after expression was switched off, compared to the time point at which doxycycline was removed.

α-Synuclein is Degraded by the Proteasome in our Cell Model

α-Synuclein expression was switched on for 24 hours, then doxycycline was removed and the proteasome inhibitors epoxomicin or lactacystin added for 48 hours. We consistently saw an increase in α-synuclein levels with treatment with the proteasome inhibitors epoxomicin or lactacystin, in both cycling and differentiated cells in all cell lines. Addition of these drugs did not cause increased cell death or overt aggregate formation.

α-Synuclein is Degraded by Autophagy

3Methyladenine (3MA) inhibits autophagy at the sequestration stage, where a double membrane structure forms around a portion of the cytosol. Bafilomycin A1 (baf A1) is a vacuolar ATPase inhibitor that interferes with the autophagosome-lysosome fusion step and rapamycin is an antifungal macrolide antibiotic that stimulates autophagy.

PC12 cell lines as described above (i.e. mitotic and neuronally differentiated wild-type, A53T and A30P PC12 lines) were induced for 24 hours, then doxycycline was removed and the drugs were added for 48 hours. The trends we observed were similar in cells treated with drugs for 24 and 72 hours.

Rapamycin, which stimulates autophagy, was observed to significantly reduce the levels of all three forms of α-synuclein in both cycling and neuronally-differentiated PC12 cells. In other words, the clearance of α-synuclein from the cells was enhanced by rapamycin.

Inhibiting autophagy with 3MA and baf A1 led to an obvious increase of α-synuclein levels in the A53T lines. These inhibitors only induced slight changes, at best, in α-synuclein levels in mitotic wildtype and A30P lines (e.g. 3MA with wildtype). In differentiated cells, the A53T lines again showed greater susceptibility to accumulating α-synuclein after treatment with these inhibitors. The densitometry values of α-synuclein bands (as a function of tubulin) were compared for 3MA and baf A1 treatments to those for control cells and calculated the fold increase in α-synuclein levels in the cells after drug treatment. Both inhibitors had significant effects on A30P accumulation (A30P+3MA: 1.13 fold increase (+/−0.04 SE); p=0.04 (n=4 experiments; paired t tests in all cases); A30P+baf A1: 1.19 fold increase (+/−0.015 SE); p=0.006 (n=3)). While the trend for wildtype α-synuclein provides indication that these inhibitors were impairing its degradation, the data did not reach significance (wt+3MA: 1.48 fold increase (+/−0.36 SE); p=0.26 (n=5); wt+baf A1: 1.12 fold increase (+/−0.08 SE); p=0.23 (n=4)).

α-Synuclein is Seen in Vesicles with Autophagic Morphology

The subcellular localisation of α-synuclein in the cell lines was examined using immunogold electron microscopy. An antibody to the HA-tag in the α-synuclein transgene products was used to detect α-synuclein. Gold α-synuclein labelling was apparent over vesicles with autophagic morphology i.e. the vesicles had morphologies consistent with those described in previous studies of autophagy (Mizushima, N., et al. (2001) J. Cell Biol. 152, 657667), Huntington's disease (Kegel, K. B. et al (2000) J. Neurosci. 20, 72687278) and α-synuclein (Stefanis, L. (2001) J. Neurosci. 21 95499560). (In these studies, vesicles were identified as autophagic on similar morphologic grounds, in the absence of specific antibodies).

α-synuclein was observed either “free” or associated with electron dense bodies of around 100 nm in diameter. There were often 2 or more gold particles over these bodies, which could indicate α-synuclein microaggregates. Such bodies were also seen in the cytoplasm, sometimes associated with α-synuclein.

These electron dense bodies may be dense core granules transporting neurotransmitters. Confocal immunofluorescence studies showed that α-synuclein (wildtype, A30P and A53T) was localised inside acidic vacuoles labelled using LysoTracker Red. LysoTracker Red labels acid compartments including degradative autophagic vacuoles and lysosomes. α-synuclein was not excluded from these compartments and speckles of α-synuclein immunoreactivity are clearly visible within these vacuoles.

mTOR is sequestered into huntingtin aggregates

Immunocytochemistry was performed using anti-mTOR antibody in COS-7 cells transiently transfected with EGFP-tagged huntingtin exon-1 fragments with 23 (Q23) or 74 (Q74) glutamine repeats. mTOR was observed to be diffusely distributed throughout the cell in untransfected cells, cells expressing the wild-type protein and in cells expressing the mutant protein without aggregates. However, in Q74-expressing cells with aggregates, mTOR was observed to colocalised- with both cytoplasmic and nuclear aggregates in >98% of cells with aggregates. The high rate of co-localisation observed with mTOR is much greater than with most other proteins analysed (Wyttenbach, A. et al. Proc Natl Acad Sci USA 97, 2898-2903 (2000)).

mTOR aggregation was never seen in the absence of huntingtin aggregates. Similar results were obtained with a phospho-specific mTOR antibody (anti-p-mTOR (Ser 2448)). The phospho-mTOR immunoreactivity appeared to be localised to the surface of the aggregates, while the total mTOR antibody stained the entire aggregate. This may be because the antibodies have different epitopes, which have different accessibility in the aggregates, or the phosphorylated form of mTOR is found in different parts of the aggregate compared to non-phosphorylated forms. mTOR sequestration was not an artefact of protein overexpression or aggregation, since no colocalisation of certain upstream mTOR regulators or unrelated proteins was observed with Q74 aggregates.

To determine whether mTOR was sequestered in huntingtin aggregates in vivo, immunohistochemistry was performed on transgenic mice expressing N-terminal mutant huntingtin fragments (Schilling, G. et al. Hum Mol Genet 8, 397-407 (1999)) and Grade III HD post-mortem brain tissue. The mTOR antibody gave specific bands of the correct molecular weight in brain lysates from control mice. Huntingtin aggregates were labelled with anti-huntingtin (EM48) or anti-ubiquitin antibodies. Double-labelling immunofluorescence in transgenic mice with EM48 and anti-mTOR antibodies showed that mTOR colocalised with huntingtin aggregates. Similar results were obtained using Avidin: Biotinylated enzyme Complex (ABC) detection

Colocalisation of mTOR in huntingtin aggregates was also found in the caudate and putamen of Grade III HD brains, but not in age-matched control brains.

Biochemical analyses was performed on cell lysates from COS-7 tells transiently transfected with EGFP-tagged Q23/Q74 or PC12 cells stably expressing EGFP-tagged Q23/Q74. Western blots of whole cell lysates revealed significant amounts of mTOR (normally a 289 kDa protein) as an abnormally slow migrating high molecular mass product in the stacking gel, characteristic of aggregates in Q74 but not Q23 expressing cells. The presence of mutant huntingtin in the stacking gel has been a criteria used by many labs for its insolubility (Waelter, S. et al. Mol Biol Cell 12, 1393-1407 (2001)). This higher molecular mass product in the stacking gel was also detected using anti-p-mTOR (Ser2448) antibody and an anti-GFP antibody directed to the huntingtin transgenes

Note that a significant amount of endogenous mTOR in the mutant lines is sequestered into the insoluble fraction compared to the soluble mTOR. Lysates from these cell lines were blotted with numerous antibodies (>20), including antibodies to hsp70, hdj1, CBP and other proteins that colocalise with aggregates. There was no sign of the obvious accumulation of these proteins in the stacking gels that was observed with mTOR. Thus, this may be a very tight interaction. These results, together with the immunofluorescence data, provide indication that mTOR is recruited into huntingtin aggregates.

To test whether the mutant huntingtin fragment interacts with mTOR, lysates from stable inducible wild-type or mutant PC12 lines and COS-7 cells transiently transfected with Q23 or Q74 were immunoprecipitated with anti-EGFP antibody and western blots were probed with anti-mTOR antibodies. Mutant huntingtin exon-1 coimmunoprecipitated mTOR mostly as high molecular mass aggregates in the stack. The difference in the mobility of these high molecular weight immunoprecipitates in the PC12 and COS-7 cells may reflect the greater heterogeneity in aggregates seen in the transiently transfected vs. stable inducible cells. This is not a generic feature of Q74, as the same type of immunoprecipitation does not pull down other proteins like S6K1 and 4E-BP1. Mutant huntingtin fragment from transgenic mouse brain lysates were also found to interact with mTOR, when immunoprecipitated with anti-mTOR antibody and immunodetected with IC2 antibody that recognises the expanded polyQ tract. These results provide indication that mutant huntingtin fragment can interact with mTOR and thus can be recruited into aggregates. This interaction requires an expanded polyglutamine tract, since the wild-type protein with 23 glutamine repeats does not associate with mTOR. Thus, we have shown that mutant but not wild-type huntingtin physically interacts with mTOR by immunoprecipitation, colocalises with huntingtin aggregates as judged with two different antibodies, and gets stuck in the stacking gel in the same place as mutant huntingtin. Furthermore, mTOR never aggregates in the absence of huntingtin aggregates in cells expressing Q74. Thus, mTOR is sequestered into huntingtin aggregates.

We next looked for changes in the levels of soluble mTOR in Q23 and Q74 expressing cells and HD transgenic mouse brains. Reduced levels of soluble mTOR (as a function of tubulin) were found in stable inducible cell lines (FIG. 5) expressing the mutant protein compared to wild-type and in HD transgenic mouse brain lysates compared to control littermates (FIGS. 6A & B). These results provide indication that sequestration of mTOR with mutant huntingtin aggregates contributes to reduced levels of soluble mTOR.

Sequestration of mTOR into aggregates would also impair nucleo-cytoplasmic shuttling of mTOR, which is crucial for its activity (Kim, J. E. & Chen, J. Proc Natl Acad Sci USA 97, 14340-14345 (2000)).

The HD Mutation Impairs mTOR Kinase Activity

mTOR phosphorylates at least two downstream substrates namely translation initiation factor eIF-4E binding protein (4E-BP1) and ribosomal protein S6 kinase-1 (S6K1) (Fingar, D. C. et al Genes Dev 16, 1472-1487 (2002)). 4E-BP1 and S6K1 are important regulators of cap-dependent and terminal oligopyrimidine tract (TOP) -dependent translation, respectively.

The cellular distribution of 4E-BP1 and S6K1 was examined in cell lines and HD grade III brains. 4E-BP1 was diffusely distributed in wild-type cells and colocalised with mutant huntingtin aggregates in ˜30% of cells with aggregates. 4E-BP1 was also found in the nuclear inclusions of HD brain tissue (caudate and putamen regions). The colocalisation of 4E-BP1 with huntingtin aggregates but its failure to be immunoprecipitated by mutant huntingtin may reflect weak or transient interactions. Unfortunately, experiments to test if S6K1 was in aggregates were not possible, since the S6K1 antibody gave no specific immunocytochemical staining above background in wild-type cells with either immunofluorescence or ABC detection methods.

In order to assess mTOR functional capacity, mTOR activity was first assessed under controlled conditions in PC12 stable inducible cell lines which show no evidence of enhanced cell death or mitochondrial dysfunction for at least 3 days after induction of transgene expression, (although increased toxicity does become apparent at 6 days after induction in cycling cells) (Wyttenbach, A. et al. Hum Mol Genet 10, 1829-1845 (2001)). After 48 h induction of the transgene expression when the following experiments were performed, there was no evidence of ER stress in either wild-type or mutant lines as judged by BiP levels on western blots. The kinase activity of mTOR can be inferred by changes in the phosphorylation status of S6K1 and 4E-BP1 (Schmelzle, T. & Hall, M. N. Cell 103, 253-262 (2000)). Reduced levels of endogenously available phosphorylated 4E-BP1 and S6K1 were found in the mutant lines, when compared to total 4E-BP1 and S6K1 respectively (FIGS. 7A and 7B). A mean reduction of 31% (SE+/−4.3) in p-S6K1 and a mean reduction of 30% (SD+/−4.5) in p-4E-BP1 were found in our mutant lines compared to wild-type cells. Note that the multiple bands for 4E-BP1 are a known consequence of its differently phosphorylated species.

The ability of mTOR in wild-type and mutant PC12 cells to phosphorylate transiently-transfected FLAG tagged 4E-BP1 was also measured (Kim, D. H. et al. Mol Cell 11, 895-904 (2003)). PC12 cells stably expressing Q23 and Q74 were transiently transfected with FLAG-tagged 4E-BP1 and serum starved for 24 h after 2 days of transfection (with simultaneous induction of the transgene). At this time point more than 99% of the mutant PC12 cells form insoluble Q74 aggregates (Wyttenbach, A. et al (2001) supra). Following starvation, the cells were stimulated with 10% FBS for 1 h before processing for immunoprecipitation.

Reduced phosphorylation of FLAG-4E-BP1 compared to total FLAG-4E-BP1 was consistently found in the mutant (Q74) vs. wild-type (Q23) cells (FIG. 7C), providing indication that endogenous mTOR in Q74 show impaired ability to phosphorylate transiently transfected 4E-BP1.

As with all data reported here, results have been confirmed in two independent sets of Q23 and Q74 clonal lines. These results demonstrate that immobilisation of mTOR in huntingtin aggregates can impair the signalling to its downstream targets. No change in the levels of phosphorylated Akt compared to total was observed between wild-type and mutant cells, providing indication that the reduced phospho-4E-BP1 and phospho-S6K1 is not the result of generic kinase inhibition. In order to test if phospho-S6, the target of activated, phosphorylated S6K1, was specifically reduced in cells with aggregates, we used an assay described by Kim and colleagues (Kim, D. H. et al. Mol Cell 11, 895-904 (2003)) to analyse the phosphorylation status of S6 protein by immunofluorescence in single COS-7 cells expressing wild-type and mutant huntingtin exon-1.

COS-7 cells expressing Q23/Q74 were immunostained for phospho-S6 (Ser235/236). ˜26% (SE+/−2.6) of cells with Q74 aggregates were found to have a negative staining for the S6-P when compared to 8.5% (SE+/−1.2) and 3.8% (SE+/−0.5) in Q23 expressing cells and Q74 expressing cells without aggregates, respectively (FIG. 8). Note that identical conditions were used for all slides and comparisons of Q74 cells with and without aggregates were performed on the same slides. Similar results were obtained using huntingtin exon-1 constructs with 25 (Q25) or 103 (Q103) glutamine repeats (FIG. 9). Double immunofluorescence analysis was performed with anti-huntingtin (EM48) and anti-S6-P antibodies in HD transgenic mouse and control brains. Consistent with the cell line data, cells with aggregates were found to stain less brightly for the S6-P antibody compared to non-aggregate containing cells or controls. About 60% of cells with aggregates showed marked reduction in S6-P immunoreactivity, while no obvious reductions were seen in control brains (<2% cells). Since cell death is not an obvious feature in these HD mice (Schilling, G. et al. Hum Mol Genet 8, 397-407 (1999)), these findings cannot be a consequence of apoptosis.

The HD Mutation Impairs mTOR-Dependent Regulation of Translation

Phosphorylation of the ribosomal protein S6 by S6K1 positively regulates the translation initiation of mRNAs containing 5′-terminal oligopyrimidine tracts (5′-TOP), like mRNAs coding for ribosomal proteins and translation elongation factors. A previously validated luciferase reporter assay was used to measure mTOR-dependent TOP-translation (Kim, J. E. & Chen, J. Proc Natl Acad Sci USA 97, 14340-14345 (2000), Schwab, M. S. et al. Mol Cell Biol 19, 2485-2494 (1999)). Rapamycin is a specific inhibitor of mTOR. Comparison of cells for TOP-dependent translation with this assay in the presence or absence of rapamycin allows assessment of the amount of functional cellular mTOR that can be inhibited by rapamycin. In FIG. 10C, each bar represents the difference between the luciferase activities (corrected for transfection efficiency-see methods) in the presence vs. the absence of rapamycin, for a luciferase reporter with a TOP sequence (+TOP) (FIG. 10A) and otherwise identical control construct without the TOP sequence (−TOP) (FIG. 10B). Rapamycin significantly inhibited the TOP-dependent (+TOP) expression of luciferase in the Q23-expressing cells after serum stimulation by about 18%, similar to previous studies with this assay in normal cells (FIG. 10C) Kim, J. E. & Chen, J. (2000) supra, Schwab, M. S. et al (1999) supra). This modest effect is because mTOR only partially controls this process. However, rapamycin had no such inhibitory effect in the Q74-expressing cells (FIG. 10C). As expected, rapamycin had no inhibitory effect on luciferase activity in Q23 cells transfected with β-gal and an-otherwise identical control-luciferase construct without the TOP sequence (−TOP) (FIG. 10B). The failure of rapamycin to downregulate TOP-dependent translation in Q74-expressing cells provides indication that mTOR activity is substantially reduced as a function of its sequestration to the huntingtin aggregates.

Consistent with these data, the raw luciferase data with the +TOP constructs (FIG. 10A) showed lower values for the Q74 cells in the absence of rapamycin compared to the Q23 cells. The increase in TOP-independent translation in the Q74 lines in FIG. 10C is compatible with data seen in other cell models where these assays were used. This was previously commented on and was attributed to competition between different classes of mRNA for the cellular translation machinery. However, the key issue in this figure is the TOP-dependent translation (not the TOP-independent data). The TOP-dependent effects in FIG. 10C were confirmed in FIG. 11 where similar results were obtained in COS-7 cells transiently transfected with Q23/Q74 along with another set of luciferase reporter constructs with a non-typical TOP sequence that was also normally responsive to rapamycin treatment (FIG. 11C). The data in FIG. 11C is represented as explained above for FIG. 10C. Cells transfected with Q23 showed a similar rapamycin-dependent reduction (23%) in luciferase activity to what was previously described, but no such effect was seen with cells transfected with Q74, which behaved like cells expressing non-functional mTOR mutants. Again, and consistent with the other data in FIGS. 10 and 11, it was noted that the raw luciferase data in FIG. 11A with the +TOP constructs showed lower values for the Q74 cells in the absence of rapamycin, compared to the Q23 cells (FIG. 11A). Together with S6 phosphorylation data; these results suggest that there is dysregulation of translation in mutant huntingtin expressing cells as a result of impaired mTOR signalling to its downstream effectors.

mTOR Sequestration Associated with Decreased mTOR Activity is Seen in Other polyQ Diseases

Sequestration of mTOR was tested for other polyglutamine diseases. The huntingtin exon-1 constructs used previously formed mostly cytoplasmic aggregates (Q74-90%; Q103-98%; note that the rate of aggregate formation is time dependent and varies with different lengths of polyQ). In order to test if mTOR signalling was affected by nuclear aggregates, double immunofluorescence was performed with anti-mTOR and anti-Xpress antibody in COS-7 cells transiently transfected with ataxin 1 constructs encoding the spinocerebellar ataxia type 1 protein with 2 or 92 polyglutamine repeats which are expressed predominantly-in the nucleus. mTOR was found to colocalise with ataxin 1 aggregates. mTOR also colocalised with aggregates formed by isolated polyQ stretches fused to GFP (Q81) and aggregates found in brains of SCA 2, 3, 7 and DRPLA patients.

A correlation of negative staining was also observed for phospho-S6 in aggregate-containing cells in COS-7 cells transfected with ataxin 1 and with Q81 (FIG. 12), providing indication that these results might be polyQ-dependent irrespective of location. Thus, impaired mTOR signalling may play a role in other polyglutamine diseases.

The HD Mutation Induces Autophagy

LC3-II is a form of microtubule associated protein-1 light chain-3 (MAP-LC3), which is associated with mammalian autophagosomes (Kabeya, Y. et al. Embo J 19, 5720-5728 (2000)). LC3-II has been found to be associated with the autophagosomes in several cell types and its levels (compared to actin) allow quantitation of autophagosome number. Western blot analysis on lysates from PC12 cells expressing either the wild-type or the mutant protein for 48 hours showed increased levels of LC3-II in our mutant lines compared to the wild-type as a function of actin (FIG. 13). While this effect was subtle, it was consistent and reproducible in four independent experiments. In addition, LC3-II increased upon induction of mutant huntingtin fragment expression in the stable PC12 cells, while no difference was seen upon induction of the Q23 cells. Another way one can score autophagic activity is to assess the number of LC3-stained vesicles. A marked increase in the number of LC3-stained vesicles was observed upon serum starvation or rapamycin treatment (which inhibits mTOR), which are known to stimulate autophagy. Furthermore, immunofluorescence analysis of COS-7 cells expressing Q23 or Q74 with anti-LC3 antibody shows increased autophagic vacuoles in cells expressing Q74 with aggregates (FIG. 14).

The same effects were confirmed with another set of HD exon-1 constructs, where increased autophagic vacuoles were seen with Q103 with aggregates but not with Q25 (FIG. 15). The predominant increase in autophagic vesicles in aggregate-containing cells is consistent with reduced S6 staining in aggregate-containing cells, since both these pathways are regulated by mTOR.

The association of mTOR with polyglutamine aggregates and the impaired mTOR signalling observed in HD models is not a secondary consequence of cell death. These phenomena were observed in a PC12 stable inducible model at a time point where cell death was negligible in the mutant lines and similar to the wild-type lines (Wyttenbach, A. et al. Hum Mol Genet 10, 1829-1845 (2001)). Furthermore, cell death is not an obvious feature of the HD mice studied (Schilling, G. et al. Hum Mol Genet 8, 397-407 (1999)).

The results herein show that mTOR is sequestered to mutant huntingtin aggregates and that this is associated with decreased S6 phosphorylation-and increased autophagy in cells with non-apoptotic nuclear morphologies and in cells treated with the pan-caspase inhibitor z-VAD-fmk.

Increasing mTOR Activity Enhances Polyglutamine Toxicity In Vitro

The cell model experiments herein show that autophagy induced by a reduction in mTOR activity reduces the levels of mutant huntingtin and related aggregate-prone proteins, and this reduces aggregate formation and cell death, when rapamycin is used early. Activation of mTOR signalling may thus enhance mutant huntingtin aggregation and toxicity.

Overexpression of the small G protein rheb greatly enhances mTOR signalling and can even activate mTOR in the absence of growth factors and amino acids (Manning, B. D. & Cantley, L. C. Trends Biochem Sci 28, 573-576 (2003)). The effects of rheb overexpression were tested in cells expressing either Q74 or Q103 constructs (FIGS. 16 and 17). In both cases, rheb overexpression (compared to transfection of an equivalent amount of empty vector control DNA) resulted in a dramatic increase in both huntingtin aggregate formation and cell death.

Rapamycin Treatment Reduces Neurodegeneration in a Drosophila HD Model

A Drosophila HD model that expresses the first 171 residues of huntingtin with 120 glutamines (120Q) in photoreceptors (Jackson, G. R., et al. Neuron 21, 633-642 (1998)) was used to test whether long-term mTOR inhibition with rapamycin would have beneficial effects in vivo. The mutant flies show photoreceptor neurodegeneration, a phenotype that is easily quantifiable using the pseudopupil technique (Franceschini, N. & Kirschfeld K. Kybernetik 9, 159-182 (1971)). No degeneration is seen in flies expressing analogous constructs with 20Q (wild-type transgenics). The compound eye of Drosophila comprises many ommatidia. An ommatidium normally contains eight photoreceptor neurons with light gathering parts called rhabdomeres, seven of which can be easily visualized using the pseudopupil technique. The number of visible rhabdomeres in each ommatidium decreases as a function of time when there is photoreceptor neurodegeneration, like in the 120Q fly lines.

Drosophila with 120Q were treated with 1 μM rapamycin or its carrier DMSO (as a control) starting in the larval stage and continuing into adulthood. This dose of rapamycin (that was previously shown to effectively inhibit Drosophila TOR function: Zhang, H. et al Genes Dev 14, 2712-2724 (2000)) dramatically rescued 120Q-induced photoreceptor degeneration (FIGS. 18 and 19). Rapamycin significantly increased the number of visible rhabdomeres per ommatidium in flies analysed at both two and three days after eclosion (hatching from the pupa) (p<0.0001; Mann-Whitney U test). At these times, there is no reduction in the number of visible rhabdomeres in wild-type untreated flies.

Decreasing mTOR Activity Reduces Severity of Symptoms in an HD Mouse Model

Rapamycin has poor water solubility and stability in aqueous solution and rapamycin ester analogues such as CCI-779 have been designed (Huang, S. & Houghton, P. J. Drug Resist Updat 4, 378-391 (2001)). CCI-779 has favourable pharmaceutical properties compared to rapamycin, has anti-tumor activity but induces only mild side effects in patients and is in Phase II and Phase III clinical trials for cancer treatment (Elit, L. Curr Opin Investig Drugs 3, 1249-1253 (2002)). For these reasons, and because this drug has been specifically shown to decrease mTOR activity in neurons (Kwon, C.-H. et al Proc Natl Acad Sci USA 100, 12923-12928. (2003)), CCI-779 was used in an HD mouse model.

CCI-779 was shown to enhance the clearance of mutant huntingtin exon-1 fragments in a cell model. A stable doxycycline-inducible PC12 cell line expressing EGFP-tagged HD exon-1 with Q74 was used, which we have previously characterised (Schwab, M. S. et al (1999) supra) This cell line allows transgene expression to be specifically switched off by removing doxycycline from the medium, without interfering with ongoing cellular protein synthesis. The stable lines were induced for 8 hours and expression was then switched off by removing doxycycline from the medium for the next 24, 48 or 72 hours, (24, 48 or 72 h OFF). CCI-779 treatment enhanced the clearance of the mutant huntingtin transgene in these experiments as judged by western blotting end also by fluorescence of the GFP-tagged transgene product (FIG. 20). This was associated with enhanced clearance of aggregates.

The efficacy of CCI-779 treatment on an HD mouse model was assessed by measuring performance on four different phenotypes that have been found to reliably discriminate between these HD mice and their wild-type littermates: rotarod performance, grip strength, wire manoeuvre and tremors (Rogers, D. C. et al. Mamm Genome 8, 711-3 (1997)). The overall performances on each of these tasks over the course of the CCI-779 trial was significantly improved in CCI-779-treated HD mice, compared to placebo-treated littermates with the same mutation (FIGS. 21 to 24). These improvements were not seen in wild-type littermates treated in parallel. There were no differences in the male:female ratios of the placebo and treated groups, nor in any of their test performances at 4 weeks of age. Weight loss is a parameter that is associated with disease in these HD mice, and has been used previously to assess potential therapeutics. However, weight is not a meaningful measure of rapamycin/CCI-779 treatment response in these mice, since CCI-779 reduces weight gain in wild-type mice (FIG. 25), compatible with previous observations with rapamycin in rodents (Walpoth, B. H. et al. Eur J Cardiothorac Surg 19, 487-492 (2001)). While brain weight was reduced in CCI-779-treated compared to placebo-treated HD mice (FIG. 26), the brain weights corrected for body weights were not reduced by this treatment, and showed a trend towards being higher in the CCI-779 group compared to the placebo group (FIG. 27).

A reduction in the density of aggregates was observed in the striatum of CCI-779-treated mice compared to placebos. Furthermore, the aggregates in the CCI-779 treated mice were small and thus sometimes difficult to see, while those in the placebo-treated mice were qualitatively larger. The difference in aggregate density induced by CCI-779 may, in fact, be greater than we perceived it to be as the overall mass (and therefore volume) of the CCI-779-treated brains is significantly less than those in the placebo controls. These changes are compatible with CCI-779 impairing mTOR activity and inducing autophagy. Impaired mTOR signaling in the brains of CCI-779 treated mice was confirmed using an antibody specific for the phosphorylated form of S6—a dramatic reduction in signal was observed in CCI-779 treated mice.

Huntingtin Aggregates Sequester and Inactivate mTOR

Mutant huntingtin is shown herein to interact with mTOR, which it sequesters in inclusions in vitro and In vivo. The sequestration of mTOR by mutant huntingtin was associated with decreased mTOR activity, assessed by impaired phosphorylation of its targets S6K1 and 4EBP1 (both endogenous and transiently-transfected). Furthermore, consistent with the decreased S6K1 phosphorylation/activity, there were decreased levels of phosphorylation of its substrate S6 in cells with aggregates, and in vivo. The reduction of mTOR activity in the cells with mutant huntingtin aggregates led to a situation where the activity of this kinase could not be further reduced by rapamycin: cells expressing mutant huntingtin fragments could not efficiently downregulate translation of constructs that were downregulated by rapamycin in cells expressing wild-type constructs. This impairment of mTOR function was seen with a construct containing a classical 5′-TOP sequence and also with construct with a non-classical 5′-UTR, both of which were normally rapamycin-sensitive. The inability of cells with aggregates to respond to rapamycin may explain the observation that this drug could not efficiently reduce huntingtin aggregate levels (via autophagy of mutant huntingtin fragments) in cell populations where about 30% were aggregate-positive, but could reduce aggregates in cells where 10% were aggregate-positive (Ravikumar, B. et al Hum Mol Genet 11, 1107-1117 (2002)).

Autophagy Induction in Cell Models

In the cell model experiments herein, polyQ and polyA expansions are used as models for aggregate-prone proteins caused by codon reiteration mutations. The results show+ that autophagy is indeed involved in the degradation of our model proteins, as these accumulated when cells were treated with different inhibitors acting at distinct stages of the autophagy-lysosome pathway, in two different cell lines. These inhibitors are used to test the role of autophagy in different contexts. Furthermore, rapamycin, which stimulates autophagy, enhanced the clearance of our aggregate-prone proteins. Rapamycin also reduced the appearance of aggregates and the cell death associated with the polyQ and polyA expansions.

The results herein also show that α-synuclein is degraded by both the proteasome and autophagy in inducible PC12 cell lines. Western blotting data showed that autophagy is a clearance route for this protein and this was confirmed by by observations of wildtype, A53T and A30P species in autophagic vesicles by EM. This is the first report that autophagy as well as the proteasome is a route. for α-synuclein degradation. Furthermore, the. autophagy inducer rapamycin is shown to increase clearance of all forms of α-synuclein.

mTOR Sequestration Induces Autophagy which is Protective in HD Models

While the net overall effect of protein aggregates may be toxic (Rubinsztein, D. C. Trends Genet 18, 202-209 (2002)), inclusions are likely to modulate numerous pathways, some toxic and some that reduce aggregate toxicity. Our data show that as more aggregates form, mTOR activity is reduced and autophagy is induced. The results herein from cell, Drosophila and mouse models of HD all provide indication that mTOR inhibition leading to enhanced autophagy has a protective effect in neurodegenerative disorders, including polyQ disorders such as HD. Inhibition of autophagy by 3-methyl adenine/dimethyl adenosine, Bafilomycin A1, and rheb overexpression which act at different stages of the process, increase huntingtin accumulation/aggregation and cell death in HD cell models. Stimulation of autophagy by rapamycin reduces such aggregation. Impaired mTOR activity and enhanced autophagy may be a factor that contributes to the late-onset nature of HD and the fact that there are frequently many surviving cells with aggregates in the brains of HD patients and cases with other polyQ diseases (Kuemmerle, S. et al. Ann Neurol 46, 842-849 (1999)). Despite the induction of autophagy, aggregates will accumulate if their production exceeds their clearance.

Our experiments with rapamycin and CCI-779 in HD Drosophila and mouse models demonstrate the therapeutic potential of mTOR inhibition for the treatment of neurodegenerative disorders, in particular, codon iteration disorders including HD and other polyQ disorders. In the mice, improvements are shown in four different phenotypes that can be attributed to neurological dysfunction in this mouse model, where the transgene expression is almost exclusively neuronal in the CNS. This range of phenotypes reported is larger than most other therapeutic trials in HD mouse models.

The rapamycin/CCI-779 strategy has some advantages compared to therapeutic approaches that aim to attenuate HD by acting at downstream targets like apoptosis, reactive oxygen species or transcriptional dysregulation (Rubinsztein, D. C. Trends Genet 18, 202-209 (2002)). Gain-of-function mutations encoding intracellular toxic aggregate-prone proteins may cause disease by inducing deleterious changes in a number of parallel and distinct pathways (Rubinsztein, D. C. Sci Aging Knowledge Environ 37, PE26 (2003)). Treatment of such diseases by reducing the levels of the toxic mutant protein may be more effective than specifically trying to rescue each of the many different pathways that it perturbs (particularly since many of these may be unknown). This approach is appealing in HD since hemizygous loss-of-function of huntingtin does not cause overt deleterious effects in humans and mice.

In addition to its potential for HD, rapamycin/CCI-779 treatment may be effective in a wide range of human protein conformational and neurodegenerative diseases, since the data herein provides evidence that rapamycin can enhance the clearance of cytosolic model aggregate-prone proteins with either polyglutamine or polyalanine expansions and also various forms of α-synuclein associated with Parkinson's disease and related synucleinopathies.

1-40. (canceled)

41. A method of clearing intracellular aggregate-prone proteins in the prophylaxis of a protein conformational disorder in an individual comprising:
stimulating autophagy activity in the individual, wherein said stimulation promotes clearance of said proteins.

42. The method of claim 41 wherein the protein conformational disorder is characterised by intracellular aggregation of said proteins.

43. The method of claim 41 comprising administering an autophagy-inducing agent to said individual.

44. The method of claim 43 wherein the autophagy-inducing agent is an mTOR inhibitor.

45. The method of claim 44 wherein the autophagy-inducing agent is a rapamycin macrolide.

46. The method of claim 45 wherein the rapamycin macrolide is rapamycin.

47. The method of claim 45 wherein the rapamycin macrolide is a rapamycin analogue.

48. The method of claim 47 wherein the rapamycin analogue is selected from the group consisting of CCI-779, 40-O-(2-hydroxy)ethyl-rapamycin, 32-deoxo-rapamycin, 16-O-pent-2-ynyl-32-deoxo-rapamycin, 16-O-pent-2-ynyl-32-deoxo-40-O-(2-hydroxyethyl)-rapamycin, 16-O-pent-2-ynyl-32-(S)-dihydro-rapamycin and 16-O-pent-2-ynyl-32-(S)-dihydro-40-O-(2-hydroxyethyl)-rapamycin.

49. The method of claim 41 wherein the disorder is a codon reiteration mutation disorder.

50. The method of claim 49 wherein the disorder is a polyQ expansion disorder.

51. The method of claim 50 wherein the polyQ expansion disorder is selected from the group consisting of Huntington's disease, spinocerebellar ataxias types 1, 2, 3, 6, 7, and 17, Kennedy's disease and dentatorubral-pallidoluysian atrophy.

52. The method of claim 49 wherein the disorder is a polyA expansion disorder.

53. The method of claim 41 wherein the disorder is an α-synucleinopathy.

54. The method of claim 53 wherein the disorder is selected from the group consisting of Parkinson's disease, LB variant Alzheimer's disease and LB dementia.

55. The method of claim 41 wherein the disorder is a prion disorder.

56. The method of claim 55 wherein the prion disorder is CJD.

57. A method of identifying an agent useful in the treatment of a protein conformational disorder comprising;
contacting a mammalian cell with a test compound; and,
determining the autophagy activity of said cell,
wherein an increase in autophagy activity in the presence of said compound is indicative that the compound is a candidate agent for use in the treatment of a protein conformational disorder.

58. The method of claim 57 wherein the cell comprises a heterologous nucleic acid encoding an aggregation-prone polypeptide.

59. The method of claim 58 wherein said heterologous nucleic acid is operably linked to an inducible promoter.

60. The method of claim 59 comprising expressing said nucleic acid and stopping said expression, prior to contacting the mammalian cell with the test compound.

61. The method of claim 57 comprising modifying the compound to optimise the pharmaceutical properties thereof.

62. The method of claim 57 comprising formulating the test compound into a pharmaceutical composition.

63. A method of producing an agent for the treatment of a protein conformational disorder comprising;
modifying rapamycin to produce a rapamycin derivative; and; determining the autophagy inducing activity of said derivative.

64. The method of claim 63 comprising determining the ability of said derivative to inhibit mTOR.

65. The method of claim 64 comprising determining the ability of said derivative to enhance the clearance of intracellular aggregate-prone proteins.

66. A method of treating a polyQ expansion disorder in an individual comprising:
stimulating autophagy activity in the individual,
wherein the polyQ expansion disorder is selected from the group consisting of spinocerebellar ataxias types 1, 2, 3, 6, 7, and 17, Kennedy's disease and dentatorubral-pallidoluysian atrophy.

67. A method of treating a polyA expansion disorder in an individual comprising:
stimulating autophagy activity in the individual.

68. A method of treating an α-synucleinopathy in an individual comprising:
stimulating autophagy activity in the individual
wherein the α-synucleinopathy is selected from the group consisting of LB variant Alzheimer's disease and LB dementia.

69. A method of treating a prion disorder in an individual comprising:
stimulating autophagy activity in the individual.

70. The method of claim 66 comprising administering an autophagy-inducing agent to said individual.

71. The method of claim 70 wherein the autophagy inducing agent is an mTOR inhibitor.

72. The method of claim 70 wherein the autophagy-inducing agent is a rapamycin macrolide.

73. The method of claim 72 wherein the rapamycin macrolide is rapamycin.

74. The method of claim 72 wherein the rapamycin macrolide is a rapamycin analogue.

75. The method of claim 74 wherein the rapamycin analogue is selected from the group consisting of CCI-779, 40-O-(2-hydroxy)ethyl-rapamycin, 32-deoxo-rapamycin, 16-O-pent-2-ynyl-32-deoxo-rapamycin, 16-O-pent-2-ynyl-32-deoxo-40-O-(2-hydroxyethyl)-rapamycin, 16-O-pent-2-ynyl-32-(S)-dihydro-rapamycin and 16-O-pent-2-ynyl-32-(S)-dihydro-40-O-(2-hydroxyethyl)-rapamycin.

76. The method of claim 67 comprising administering an autophagy-inducing agent to said individual.

77. The method of claim 76 wherein the autophagy inducing agent is an mTOR inhibitor.

78. The method of claim 76 wherein the autophagy-inducing agent is a rapamycin macrolide.

79. The method of claim 78 wherein the rapamycin macrolide is rapamycin.

80. The method of claim 78 wherein the rapamycin macrolide is a rapamycin analogue.

81. The method of claim 80 wherein the rapamycin analogue is selected from the group consisting of CCI-779, 40-O-(2-hydroxy)ethyl-rapamycin, 32-deoxo-rapamycin, 16-O-pent-2-ynyl-32-deoxo-rapamycin, 16-O-pent-2-ynyl-32-deoxo-40-O-(2-hydroxyethyl)-rapamycin, 16-O-pent-2-ynyl-32-(S)-dihydro-rapamycin and 16-O-pent-2-ynyl-32-(S)-dihydro-40-O-(2-hydroxyethyl)-rapamycin.

82. The method of claim 68 comprising administering an autophagy-inducing agent to said individual.

83. The method of claim 82 wherein the autophagy inducing agent is an mTOR inhibitor.

84. The method of claim 82 wherein the autophagy-inducing agent is a rapamycin macrolide.

85. The method of claim 84 wherein the rapamycin macrolide is rapamycin.

86. The method of claim 84 wherein the rapamycin macrolide is a rapamycin analogue.

87. The method of claim 86 wherein the rapamycin analogue is selected from the group consisting of CCI-779, 40-O-(2-hydroxy)ethyl-rapamycin, 32-deoxo-rapamycin, 16-O-pent-2-ynyl-32-deoxo-rapamycin, 16-O-pent-2-ynyl-32-deoxo-40-O-(2-hydroxyethyl)-rapamycin, 16-O-pent-2-ynyl-32-(S)-dihydro-rapamycin and 16-O-pent-2-ynyl-32-(S)-dihydro-40-O-(2-hydroxyethyl)-rapamycin.

88. The method of claim 69 comprising administering an autophagy-inducing agent to said individual.

89. The method of claim 88 wherein the autophagy inducing agent is an mTOR inhibitor.

90. The method of claim 88 wherein the autophagy-inducing agent is a rapamycin macrolide.

91. The method of claim 90 wherein the rapamycin macrolide is rapamycin.

92. The method of claim 90 wherein the rapamycin macrolide is a rapamycin analogue.

93. The method of claim 92 wherein the rapamycin analogue is selected from the group consisting of CCI-779, 40-O-(2-hydroxy)ethyl-rapamycin, 32-deoxo-rapamycin, 16-O-pent-2-ynyl-32-deoxo-rapamycin, 16-O-pent-2-ynyl-32-deoxo-40-O-(2-hydroxyethyl)-rapamycin, 16-O-pent-2-ynyl-32-(S)-dihydro-rapamycin and 16-O-pent-2-ynyl-32-(S)-dihydro-40-O-(2-hydroxyethyl)-rapamycin.
